(19) United States
(12) Patent Application Publication
(10) Pub. No.: US 2020/0138625 A1
(43) Pub. Date: May 7, 2020

(54) EXOTHERMICALLY HEATED ELASTIC ADHESIVE TAPE AND WRAP WITH IMPROVEMENTS

(71) Applicant: ELECTRO-KINESIS, INC., Hatboro, PA (US)

(72) Inventors: John A. Hope, Huntingdon Valley, PA (US); Tam Van Nguyen, Philadelphia, PA (US)

(73) Assignee: ELECTRO-KINESIS, INC., Hatboro, PA (US)

(21) Appl. No.: 16/440,398

(22) Filed: Jun. 13, 2019

Related U.S. Application Data
(60) Provisional application No. 62/756,690, filed on Nov. 7, 2018.
(60) Provisional application No. 62/801,133, filed on Feb. 5, 2019.
(60) Provisional application No. 62/858,027, filed on Jun. 6, 2019.

(51) Int. Cl. A61F 7/03; A61F 13/02

(52) U.S. Cl. CPC A61F 7/034; A61F 2007/0226; A61F 13/0269; A61F 13/0243

(57) ABSTRACT

A tape 400 or wrap is provided which includes first and second elongated elastic layers 345, 346 sized to conform to the shape of a portion of the external skin of the body of a wearer; and a heated area comprising an exothermic material 310 sandwiched between said first and second elastic layers 345, 346, wherein said exothermic material 310 is activated by exposing said exothermic material 310 to oxygen; wherein said tape 400 or wrap has a elasticity of at least about 10-90% and said heated area is capable of substantially expanding and contracting with the tape or wrap. Methods of using the tape 400 or wrap are also provided.

RELATED APPLICATION DATA

This application claims priority under 35 U.S.C. § 119(e)(1) from U.S. Provisional Application Ser. No. 62/756,690, filed November 7, 2018, U.S. Provisional Application Serial No. 62/801,133, filed Feb. 5, 2019, and U.S. Provisional Application Ser. No. 62/858,027, filed Jun. 6, 2019 the contents of which are incorporated herein by reference.

### FIELD OF THE INVENTION

This invention relates to elastic therapeutic tapes and wraps and methods of treating patients with combination therapies, and more particularly to tapes and wraps containing exothermic compositions, which, after being introduced into the tape or wrap, still allow the tape or wrap stretch and recoil.

### BACKGROUND OF THE INVENTION

Elastic therapeutic tape, also called kinesiology tape, Kinesio tape, k-tape, or KT, is an elastic cotton strip with an acrylic adhesive that is used with the intent of treating pain and disability from athletic injuries and a variety of other physical disorders. The tape was invented by Japanese chiropractor Kenzo Kase in the 1970s. Strips of brightly colored tape adorning the arms, legs, and torsos of many top athletes, became world famous during the 2008 Olympics, and have increased in popularity since then.

Kinesiology tape is hypoallergenic and wearable for days at a time, for example, up to 4 days. The product is made from a type of thin, elastic cotton that can stretch up to 30%-40% of its original length. Designed to mimic human skin, with roughly the same thickness and elastic properties, it is generally latex free and includes cotton fibers which allow for evaporation and quicker drying leading to longer wear time. How the tape is claimed to affect the body is dependent on the location on the body, and how it is applied; the stretch direction, the shape, and the location. Thelen MD, Dauber JA, Stoneman PD (July 2008). “The clinical efficacy of kinesio tape for shoulder pain: a randomized, double-blinded, clinical trial”. J Orthop Sports Phys Ther. 38 (7): 389-95, which is hereby incorporated by reference herein.

Kinesiology tape was designed to run with the contours of the skin. As a result, if the tape is stretched greater than its normal length, and then adhesively applied to the skin, it will “recoil” and create a pulling force on the skin (“kinesiologic effect”), and this force microscopically lifts the skin directly beneath it to create a small interstitial space between the muscle and dermis layers. That space potentially takes the pressure off swelling or injured muscles, allows smooth muscle movement and makes space for drainage and blood flow. This elastic property also allows much greater range of motion compared to traditional white athletic tape.

With the utilization of single “I” strips or modifications in the shape of an “X”, “Y” or other specialized shapes as well as the direction and amount of stretch placed on the tape at time of application, Kinesiology tape can be applied in hundreds of ways and has the potential to reduce inflammation, prevent injury and promote good circulation and healing, and assist in returning the body to homeostasis.

While kinesiology is still a growing field and has the potential to treat many more types of injuries and conditions, there appears to be a need to provide even more functionality to standard tapes and wraps to provide greater therapeutic effect.

### SUMMARY OF THE INVENTION

In a first embodiment of the invention, a tape or wrap is provided which includes first and second elongated elastic layers sized to conform to the shape of a portion of the external skin of the body of a wearer; and a heated area comprising an exothermic material sandwiched between said first and second elastic layers, wherein said exothermic material is activated by exposing said exothermic material to oxygen; wherein said tape or wrap has a elasticity of at least about 10-90% and said heated area is capable of substantially expanding and contracting with the tape or wrap.

In further versions of this embodiment, the tape or wrap further includes a plurality of intermediate movement blocking surfaces disposed in a space formed between said first and second elongated elastic layers. The intermediate movement blocking surfaces can be formed between said first and second elongated elastic layers, so as to resist accumulating or clumping of said exothermic material at one or both ends of said tape or wrap during use.

Alternatively, the intermediate movement blocking surfaces comprise a bond formed between the first and second elongated elastic layers, or a plurality of raised or formed surfaces on the first elongated elastic layer, second elongated elastic layer, or both, or another layer, such as a grid, mesh, netting, or maze-like surface, disposed between the elongated elastic layers. The intermediate movement blocking surfaces, which help to impede the movement of said exothermic material during use, also may allow some movement of the exothermic material during use, such as the sifting of loose exothermic compound, like sand in a child's sandbox sifter, around one or more of the intermediate movement blocking surfaces, but without substantial accumulation or clumping at one or both ends of the tape or wrap.

In another embodiment of the tape or wrap, the exothermic material contained within a heated area has a first thickness when said tape or warp is unstretched and the exothermic material has a second thickness when said tape or wrap is stretched, whereby the second thickness is less than said first thickness. The exothermic material contained within said heated area and having the second thickness is capable of generating a surface temperature of about 32° C. to about 70° C. (89.6-158 ° F.).

In further embodiments, the exothermic material comprises iron powder, water, and a carbon-containing material, preferably in loose particulate form. The exothermic material can be captured in a pocket, compartment or sealed heating area, or allowed to move in the warp or weft direction, or allowed to move in the warp or weft direction with preferred intermediate movement blocking surfaces partially impeding the movement of the exothermic material.

In further versions of the tape or wrap a second of said elongated elastic layers is permeable to gaseous oxygen and resistant to liquid water, while a first of said elongated elastic layers can be perforated to provide enough oxygen in an ambient environment to permit said exothermic compound to exothermically react to generate a temperature of at least about 100 F (37.8 C) for at least about 30 minutes.

In further embodiments of this invention, first and second elongated elastic layers comprise a non-woven polymeric film having a Tensile Strength, (per ASTM D 882), of about 2000-10000 psi, a Stress, at 100% elongation, (per ASTM 882), of about 200-3000 psi, a Tear Strength, (per ASTM 624), of about 100-1000 lbf/in; and a Glass Transition Temperature (as customary) of about −100 F-+10 F.

In still a further embodiment, the tape or wrap which is sized to conform to a portion of the external skin of the body of a wearer is provided. The tape or wrap has a longitudinal length, a width and at least two transverse ends, and comprises first and second elongated elastic layers, each of said elastic layers comprising a thermoplastic polyurethane (“TPU”) layer having a thicknesses of no greater than about 0.001-1.5 mm; said first and second elastic layers comprising at least one peripheral bond and a plurality of intermediate movement blocking surfaces disposed in a space formed between said first and second elongated elastic layers. The exothermic material of this embodiment is sandwiched between the first and second elastic layers and also at least between a first pair of said plurality of intermediate movement blocking surfaces. The first pair of said plurality of intermediate movement blocking surfaces are provided to at least partially impeding the movement of exothermic material when worn by a user; and the exothermic material is activated by exposing said exothermic material to oxygen. This tape or wrap can stretch at least about 10-90% of its original length which provides for sufficient skin contact in order to optimize heat transfer. Alternatively, this tap or wrap can be stretched greater than its normal length, and then adhesively applied to the skin of a wearer, so it will recoil and create a pulling force on the skin. More preferably, this tape or wrap can stretch up to 20-70% of its original length in the warp direction. And in certain embodiments, these tapes or wraps can stretch up to about 10%-90% of an original dimension of said tape or wrap in any direction. Optionally the provided heated areas are capable of substantially expanding and contracting with the tape or wrap.

In other embodiments, at least a first one of said first and second elongated elastic layers is micro-perforated for controlling a heating temperature and a duration of an exothermic reaction of said exothermic material.

In still a further tape or wrap of this invention, an exothermic compound layer is disposed between a first and second exothermic compound sealing layer to form a heated area substantially along the length of said tape or wrap. A first exothermic compound sealing layer is bonded to a top elastic fabric layer, and a second exothermic compound sealing layer is bonded to a second elastic fabric layer, whereby the exothermic compound layer is disposed between said first and second exothermic compound sealing layers and expands substantially proportionately in length to the tape or wrap as the tape or wrap is stretched.

In a further embodiment, a tape or wrap sized to conform to a portion of the external skin of the body of a wearer includes an exothermic compound layer disposed between a pair of elastic fabric layers to form a heated area having an elasticity (hereinafter meaning: the ability of a fiber or fabric to return to its original length, shape, or size immediately after the removal of stress) of about 10%-90% in any or all directions. This tape or wrap can be further improved by making each of said pair of elastic fabric layers with a TPU film or coating having a thickness of less than about 0.02 mm. Additionally, the exothermic compound layer could be disposed between a pair of elastic fabric layers which is then disposed between a pair of breathable insulating fabric layers also having an elasticity of about 10%-90% at least in the warp direction, so that the exothermic compound layer disposed between the pair of elastic fabric layers is capable of expanding and contracting with the elasticity of the tape or warp without substantially impeding same. This embodiment can be further improved by making said pair of elastic fabric layers with a TPU layer and making said breathable insulating fabric layers with knitted or woven fabrics.

In further embodiments, the knitted or woven fabrics comprise fibers selected from the group consisting essentially of: cotton, spandex, rayon, nylon, polyester, or a combination thereof.

In still a further embodiment, an exothermically heated elastic adhesive tape is provided in which a exothermic heating material is located between two exothermic composition sealing layers or two elongated elastic layers. These layers are preferably elastic or stretchable, and the non-elastic exothermic heating material does not interfere with the elastic properties of the adhesive tape. The preferred exothermic heating material can be loose, loose but with limited mobility, fully bonded or partially bonded between the insulating layers.

In other embodiments, the heating layer does not interfere with the contouring requirements of the heated therapeutic system, for example, with the stretch and flex requirements of the heated therapeutic system.

The exothermic heating material may be compounded for specific temperatures and/or heating durations.

In further embodiments, the exothermically heated elastic adhesive tape that is breathable throughout its thickness, so that the wearer's skin can breath and sweat can evaporate through the tape. In other embodiments, exothermic heating material is activated by exposing the adhesive tape to air. The preferred tapes can remain active, regardless of the level of stretch to which the elastic adhesive tape has been subjected. Their exothermic compounds should also not interfere with the adhesive requirements of the elastic adhesive tape, e.g., they should not degrade the adhesive properties or chemistry. Some of the tapes can include materials, such as adhesives and exothermic compound combinations, which allow the adhesive to be repositioned, without similar deterioration of the adhesive. Preferred adhesive layers may or may not incorporate an adhesive pattern.

The preferred heated elastic adhesive tape can continue to operate when exposed to moisture, are preferably water repellent, and more preferably are waterproof

This tape and wraps of this invention conveniently and economically apply heat therapy to any surface area of the body. The heated therapy system utilizes an adhesive, or other fastener, to secure the system directly to the body or onto one's clothing. The preferred tapes and wraps conveniently provide for concealed pain relief and can be worn under clothing.

The exothermic heating material location should allow for therapeutically maximizing the use of the heat generated. Preferably the heat from the tape is utilized for its known therapeutic value. In other embodiments, the effectiveness of the therapeutic heating device relies on its elasticity for making and maintaining efficient skin contact. It also conveniently lends itself for adaptation to any part of the body.

In further embodiments, an upper insulating fabric layer is used to retain the heat in order to maximize, therapeutically, its effect. In other constructions, the insulation characteristics of a lower insulating fabric layer are used to minimize the potential for any negative effects on the skin that may be caused by the exothermic reaction.

The preferred exothermically heated elastic adhesive tapes can be produced in any size, shape or pattern, in strip form, or as precut strips in roll form. Heat can be generated and/or directed from a plane or in a combination of different planes. The preferred fabrics can be selected from a weave, woven, knit, non-woven fabric (film) or a combination of these fabrics, and each fabric selected can be of different weights and thicknesses. The fabric layers may be constructed with elasticity in either one or two directions (warp and weft, for example). In another example, the tape may constructed with the stretch component of the fabric insulating layers primarily in a warp direction that, upon application to the skin with stretch, will provide Kinesiology benefit when the skin lifts as the elastic tape attempts to recoil. By providing both therapeutic heat and Kinesiology benefits, the tape become a multi-modality therapeutic product.

In a further embodiment, an exothermic heating material, that is enclosed within an envelope, such as a friable or tearable air impermeable layer, or partially perforated bag, is incorporated within an elastic adhesive tape construction, without substantially restricting the elasticity of the tape.

Also provided is a method of treating a wearer for muscle pain or injury, comprising: providing an elastic tape sized to conform to a portion of the external skin of the body of said wearer, comprising: an exothermic compound layer disposed between a pair of elastic fabric layers having an elasticity of about 10%-90% in any or all directions, and an adhesive backing layer; stretching said elastic tape about 10% -90% of its original length; adhering said adhesive backing layer of said adhesive tape to the skin of a wearer proximate to said muscle pain or injury while said elastic tape if stretched; substantially simultaneously providing heat therapy and a kinesiologic effect on said skin of said wearer proximate to said muscle pain or injury.

In another preferred embodiment of this invention, an elastic therapeutic wrap or tape is provided, which is adapted for application to the external skin of a wearer. The tape or wrap comprises: an elongated elastic fibrous layer sized to conform to a portion of the external skin of the body of said wearer; and an encapsulated exothermic compound partially adhered to the elastic fibrous layer, which compound can be activated by exposing the exothermic compound by breaking the weak, intermittently adhered or bonded, or scored barrier film, e.g. by stretching the tape or wrap to break the seal, or by unwrapping the air tight packaging or both.

In more preferred embodiments, the exothermic compound is sealed within a friable or tearable polymeric layer that is broken during use so as to expose the compound to air or oxygen so that the exothermic reaction can begin or resume.

In other embodiments, the encapsulation layer is porous to air or oxygen, such as a porous fabric like cotton or rayon, but the packaging, such as a plastic bag, for the tape or wrap, is made to be air tight, or oxygen impermeable, so that when the packaging is removed, the exothermic reaction begins or resumes.

The overall construction design, fabric selections and exothermic heating materials can enhance the systems financial competitiveness over alternative heating systems.

All documents cited herein, including publications, patent applications, and issued patents mentioned herein, are, in relevant part, incorporated herein by reference. Citation of any document is not an admission regarding any determination as to its availability as prior art to the present invention.

### BRIEF DESCRIPTION OF THE DRAWINGS

The accompanying drawings illustrate preferred embodiments of the invention as well as other information pertinent to the disclosure, in which:

FIG. 1: is an exploded front perspective view of a tape embodiment of this invention;

FIG. 2: is a top planar view a preferred tape embodiment of FIG. 1, after pealing back the top upper elastic insulating fabric layer 50;

FIG. 3: is an exploded front perspective view of another tape embodiment of this invention;

FIG. 3a: front perspective view of an elastic mesh for holding exothermic material;

FIG. 3b: is an enlarged view of a portion of the elastic mesh for holding exothermic material taken from the area denoted in FIG. 3a;

FIG. 4: is a top planar view of an alternative tape embodiment of this invention, with its top fabric layer removed;

FIG. 5: is a side elevation, cross-sectional view of the tape embodiment of FIG. 4, with the top fabric layer on.

FIG. 6: is an exploded partial front perspective view of another tape embodiment of this invention having layered shingles;

FIG. 7: is a top planar view, of an alternative tape embodiment of this invention having layered shingles, after the top layer of fabric has been removed;

FIG. 8: is a side elevation, cross-sectional view of the tape embodiment of FIG. 7, with the top fabric layer on;

FIG. 9: is a top planar view, of the tape embodiment of this invention having layered shingles of FIG. 7, after the top layer of fabric has been removed, and the tape has been stretched;

FIG. 10: is a side elevation, cross-sectional view of the stretched tape embodiment shown in FIG. 9, with the top fabric layer on;

FIG. 11: is a right side elevation view of a further tape embodiment of this invention shown in FIG. 12;

FIG. 12: is a top planar view of a further tape embodiment of this invention;

FIG. 13: is a bottom, side elevation, view of the tape embodiment shown in FIG. 12;

FIG. 14: is an exploded front perspective view of the tape embodiment shown in FIG. 12, before welding the peripheral and intermediate bonds;

FIG. 15: is a top planar view of a further embodiment of an elastic exothermic containment envelope (without exothermic material) of this invention showing formed trays or pockets; and

FIG. 16: is a side, plan, cross-sectional view of the elastic exothermic containment envelope taken through line 16-16 of FIG. 15.

### DETAILED DESCRIPTION OF THE INVENTION

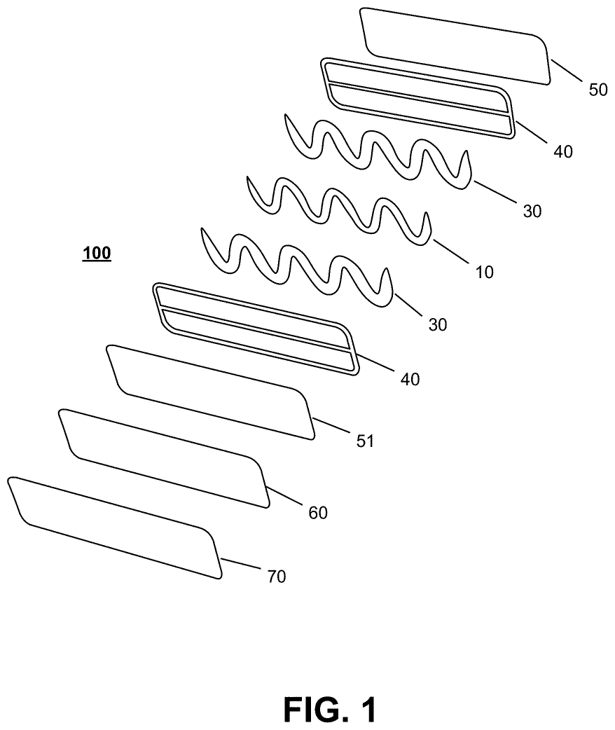

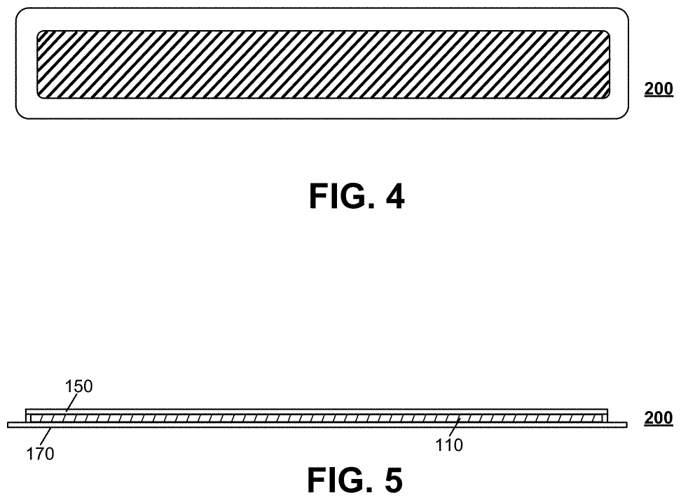

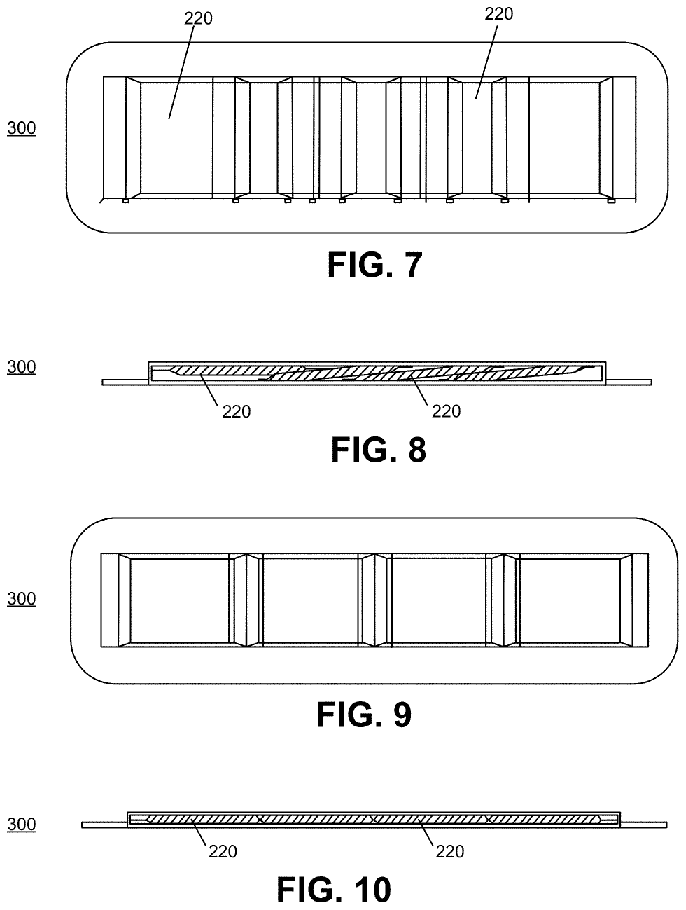

As shown in FIGS. 1-16, the preferred exothermically heated tapes 100, 200, 300 and 400 and wraps are generally precut strips are made in a variety of widths, lengths and configurations. Depending on the brand, strip length varies from 5-12″ and width from 1-4″. The most common configurations are X, Y and I, as well as precut edema strips, which are specially designed to relieve swelling, edema and lymphedema. When the exothermic compound is incorporated in a wrap, it preferably does not utilize an adhesive backing to secure the system to the body. Its ability to stay in place during therapy can rely on it being mechanically secured to the body. This securement can be made with replaceable and removable adhesive pads which can be applied to the underside of the wrap and then adhered to the skin of a wearer, an adhesive tape applied on the wrap and then to the wear's skin, elastic or mechanical straps, hook and loop materials, compression wear or some other mechanical means. In certain other embodiments, the adhesive pads can include a transdermal active, or the adhesive pads can be substituted with adhesively applied hook and loop fastener pads (one of which can be applied to the skin of the wearer and may also contain an optional transdermal active).

The preferred exothermically heated wrap or tape 100, 200, 300 and 400 is preferably, in appearance and feel, not substantially different than a wrap or a strip of kinesiology tape without the exothermic compound. The wrap or tape is able to flex, stretch and contour to the skin in substantially the same way as unheated fabric does.

### Tape Embodiments Generally

The unique construction of the preferred exothermically heated elastic adhesive tapes 100, 200, 300, and 400 and wraps of this invention is such that, because they are elastic, and able to stretch, flex and contour to the skin, they allow the heat generated within the tape, to remain against or near the skin as the body moves. The exothermic material is insulated on the top side (non-skin side) by a layer of thermally insulating elastic fabric 50, 51, 135, 150, 235, 250, 350, 351 such as woven cotton or neoprene fabric or material, in order to minimize heat loss to the atmosphere, insulate heat loss, or reflect heat either radiantly or conductively back to the wearer.

Additionally, the exothermic compound 10, 110, 210, 310 or material can be insulated on the underside of the reactive material by another layer of thermally insulating elastic fabric 51, 135, 235, 351 that is designed to protect the skin from any potential for adverse effects of the exothermic reaction, such as hot spots, while allowing the heat to penetrate through to the wearer. This lower layer of thermally insulating elastic fabric 51, 135, 235, 351 preferably conducts some heat and limits the upper temperature to which the wearer is exposed, but optionally, can also spread heat more uniformly, such as when a carbon or graphite impregnated layer is used, such as a printed carbon layer on a fabric or a fabric which has been immersed in a carbon containing resin or coating. The upper and lower layers should have stretch properties, of at least about 10-30% elasticity at the low end, for flexible and drapeable bandages and tape, to as high as 30%-90%, preferably about 65% elasticity, at the upper end, for kinesiology tape and ace bandage type wraps.

Because the heat is preferably generated utilizing an exothermic compound 10, 110, 210, 310, there need be no wires or conductive metal yarns used to convey power or used to form a heating element. It is, therefore, preferably a wireless system.

The exothermic heating area of embodiments 100, 200, 300 and 400 is preferably about 0.5-1.5″, preferably about 1″ in width, and about 5-20″, preferably about 9″, in length. The overall thickness of the tape embodiments 100, 200, 300 and 400, is about .06-.5″, preferably about 0.125-0.25″.

Additionally, no external temperature controlling device is required, as temperature control is accomplished via the compounding process of the exothermic material, alone, or in the selection and construction of the fabrics, or films used. Likewise, the heating duration can be determined by the compounding process of the exothermic material or in the selection and construction of the fabrics, or films used.

The exothermically heated elastic adhesive tape 100, 200, 300 and 400 can be similar in appearance to elastic adhesive tape without exothermic heating.

The preferred tape embodiment is able to flex, stretch and contour to the skin in a similar way as unheated adhesive tape. And, if the stretch component of the thermally insulating elastic fabric layers 50, 51, 135, 150, 235, 250, 350, 351 , and the elongated elastic layers 345 and 346, is primarily in the warp direction, or anisotropic, when applied to the skin, the recoil and skin lifting effect provides for a Kinesiology benefit, making it a multi-modality therapeutic product.

The system is preferably designed as a one-time use product. Once the exothermic reaction period is complete, the product may be removed or remain in place for added Kinesiology benefit.

The preferred tape 100, 200, 300, and 400 and wrap of some embodiments is a linear or non-linear elastic and preferably also an oxygen and water vapor porous composite material. Preferred tensile strength of kinesiology tape ranges from 80 to 180 N depending on the tape width. In a relaxed state, porosity together with air permeability and other heat and mass transfer characteristics are mainly affected by the compact layer of adhesive on the bottom surface of the tape. During tensioning, pores are expanding and therefore transfer of air and water vapor can significantly be increased and at the same time heat transfer is reduced which can positively affect wearer comfort.

The preferred tape 100, 200, 300, and 400 and wrap is hypoallergenic and wearable for days at a time, for example, up to 4 days. For kinesiology tape applications, the tape 100, 200, 300, and 400 can stretch isotropically up to 10%-90% of an original dimension in all directions, preferably about 25%-65%, or anisotropically, up to 10%-90%, preferably about 25%-65% of its original length in the warp direction and about 5-20%, preferably about 10% of its original width in the weft direction. For other applications, such as wraps, drug delivery or pads, the product can have much less stretch, and can be closer to isotropic in stretchability, such as about 1-10% in the warp direction and about 1-10% in the weft direction.

The fabric used for the tape 100, 200, 300 and 400 or wrap is preferably designed to mimic human skin, with roughly the same thickness and elastic properties as human skin. It can be generally be latex free and can sometimes preferably include fibers, film or material which allows for the evaporation of liquid water (sweat) and quicker drying leading, which can lead to longer wear time. The weight of the fabric can be about 75 gsm (when exothermic compound is later to be added), and about 160 gsm if no exothermic material is added. How the tape is claimed to affect the body is dependent on the location on the body, and how it is applied; the stretch direction, the shape, and the location. Thelen MD, Dauber JA, Stoneman PD (July 2008). “The clinical efficacy of kinesio tape for shoulder pain: a randomized, double-blinded, clinical trial”. J Orthop Sports Phys Ther. 38 (7): 389-95, which is hereby incorporated by reference herein.

The tape 100, 200, 300 and 400 is designed to run with the contours of the skin. As a result, if the tape is stretched greater than its normal length, and then adhesively applied to the skin, it will “recoil” and create a pulling force on the skin, and this force microscopically lifts the skin directly beneath it to create a small interstitial space between the muscle and dermis layers. That space potentially takes the pressure off swelling or injured muscles, allows smooth muscle movement and makes space for drainage and blood flow. This elastic property also allows much greater range of motion compared to traditional white athletic tape.

With the utilization of single “I” strips or modifications in the shape of an “X”, “Y” or other specialized shapes as well as the direction and amount of stretch placed on the tape at time of application, the preferred tape 100, 200, 300 and 400 can be applied in hundreds of ways and has the potential to reduce inflammation, prevent injury and promote good circulation and healing, and assist in returning the body to homeostasis.

Preferred properties of the tape 100, 200, 300 and 400 of this invention include: waterproof or water resistant, antimicrobial, biocompatible, up to 100% medical grade acrylic adhesive; up to 100% latex-free, hypoallergenic, does not limit range of motion, capable of multiple day wear time and the ability of applied tension on the tape to potentially relax or stimulate muscles.

In further preferred embodiments, the tape 100, 200, 300 and 400 or wrap is a relatively isotropically stretchable and made of a flexible combination of layers that is disposable, since the exothermic material is generally for single use.

In further embodiments, the exothermic compound 10 and 310 or material is sealed or sandwiched within a pair of preferred exothermic compound sealing layers 30 or elongated elastic layers 345, 346, made from a non-woven fabric, such as TPU film.

This heating unit can then be sealed by adhesively bonding, heat, sonic bonding, RF welding, or using a combination thereof, for example, and then similarly bonding the sealed heating unit to a disposable or washable fabric having a basis weight of about 75-250 grams per square meter (“gsm”), such as fabrics selected from:
3-10 wt. % spandex/90-97 wt. % cotton, polyester, rayon or nylon weaves;
100% cotton, polyester, rayon or nylon knits; or
50 wt. % nylon-50 wt. % polyester knitted blends.

Alternatively, a composite can be made by coating one or both sides of such woven or knit fabrics with an elastic polymeric layer, such as spun bonded fibers or a thermoplastic film, or a roll applied coating of thermoplastic film, such as a TPU film. Such composites can then be used as the preferred exothermic compound sealing layers 30 or elongated elastic layers 345, 346 and fabric layers 350, 351 or exothermic compound sealing layers 30 and upper and lower elastic fabric insulating layers 50 and 51.

The wraps and tapes 100, 200, 300 and 400 of this invention are designed to incorporate an exothermic compound 10, 110, 210, 310 or material. An exothermic compound generates a chemical reaction that releases energy, usually in the form of heat. The exothermic reaction occurs when certain materials are preferably exposed to oxygen. Preferably, the materials used are compounded from environmentally safe materials such as iron powder, water, water, salt, activated charcoal & vermiculite. In the preferred embodiments, at least one of the exothermic compound sealing layers 30 or one of the elongated elastic layers 345, 346 is micro-perforated to allow sufficient oxygen for the exothermic reaction to be continuous. Even if some materials for these layers are somewhat breathable in the thicknesses selected for manufacturing the tapes and wraps.

The preferred exothermic compounds 10 or 310 is desirably disposed within a pair of preferred elongated elastic film layers 345, 347 or pair of exothermic compound sealing layers 30 to form a sealed serpentine path 100, or a plurality of compartments or pockets 414 formed by a plurality of intermediate movement blocking surfaces, so that the heated area can be stretched, expanded, unwound and/or unsprung and lengthened, when the tape or warp 100, 400 is stretched and applied to a wearer by wrapping or adhering.

The tapes 100, 200, 300 and 400 should be breathable when worn, so that each of the recited layers: 30, 40, 50, 60 in tape 100, layers 140, 135, 150, 160 in tape 200, layers 225, 240, 250, 235, 260 in tape 300, and layers 350, 345, 310, 346, 351, 347, 360 of tape 400 should either be breathable (to air or oxygen, as well as water vapor) or made to be so when stretched and worn.

The manufacturing of the tapes 100, 200, 300 and 400 is preferably conducted in an oxygen free environment such as Argon or Nitrogen, or the exothermic composition 10, 110, 210 and 310 can be sealed from oxygen relatively early in its exothermic reaction.

Ideal dimensions of the exothermic material component can include a thickness of about 1/16-⅛ inches (1-4 mm) and width of about 0.25-0.50 inches (6.35-13 mm), which can then be subdivided into sections or packets in separate shingles or in seamed compartments or along the serpentine path, for example. This subdivision allows the exothermic reaction and resulting heat to be more uniformly distributed along the tape or wrap, when worn on the body vertically or horizontally, or when stretched or unstretched.

By providing heat, the wrap or tape 100, 200, 300 and 400 is providing a blood flow stimulant. When a body is warming up, prior to a work-out or exercise, increased blood flow to the muscles, bones and surrounding tissue is known to be of enormous benefit for the purpose of preventing injury. Additionally, stimulating blood flow is useful in providing pain relief and, in general, for advancing the healing of the body.

### Serpentine Embodiment:

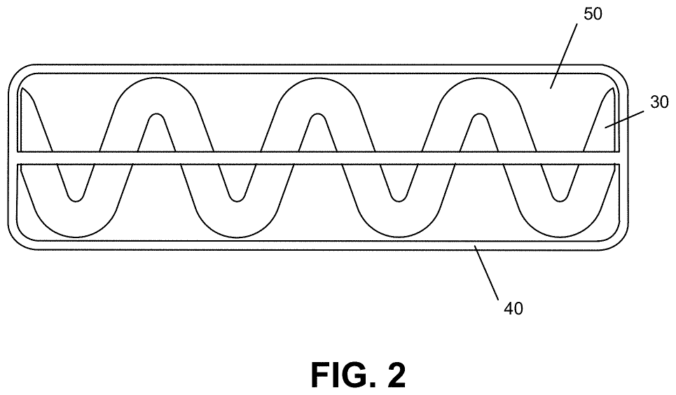

In a first embodiment of the invention, shown in FIGS. 1 and 2, a tape 100 or wrap is provided which is sized to conform to a portion of the external skin of the body of a wearer, comprising: an exothermic compound layer 10 disposed between a pair of exothermic compound sealing layers 30, said exothermic compound layer 10 and said pair of exothermic compound sealing layers 30 forming a serpentine path substantially along the length of said tape 100 or wrap, said exothermic compound sealing layers being adhered to first and second thermally insulating elastic fabric layers 50 and 51, respectively, by adhesive layers 40 disposed at least along a central axis of said serpentine path, and more preferably along the central axis and optionally, along the outer periphery of the serpentine path (so as to allow it to unwind and contract as the tape 100 unwinds and contracts) whereby the exothermic compound layer 10, disposed between said pair of exothermic compound sealing layers 30 in said serpentine path, unwinds substantially proportionately in length to the tape 100 or wrap as the tape or wrap is stretched. An adhesive layer 60 for the tape 100 can be added, along with a release liner layer 70, so the tape can be applied to the skin or clothing of a wearer. If the end product is a wrap the adhesive layer 60 and release layer 70, can be eliminated.

### Preferred Construction Sequence of the Serpentine Embodiment:

The following process preferably begins with rolls of: exothermic material which has been compounded and then sealed between a pair of exothermic compound sealing layers, a lower elastic fabric insulating layer having been nearly completely laminated with adhesive on the skin-facing side, and laminated with adhesive bands on its non-skin facing side and then protected with release liners on each of the adhesive layers, and an upper elastic fabric insulating layer which has been laminated with adhesive on one side and then covered by a respective release liner.
1. Load compounded exothermic material into compound retaining sleeve with exothermic material.
2. Pre-form loaded exothermic compound sleeves for thickness, width and serpentine path pattern.
3. Laminate pre-formed exothermic compound sleeves to adhesive band(s) on the non-skin side of the lower elastic fabric insulating layer.
4. Laminate adhesive band(s) of the upper elastic fabric insulating layer to the exothermic material side of the above assembly.
5. Die-cut assembly to final size.
6. Screen graphics on upper elastic fabric insulating layer.
7. Seal completed assembly in airtight packaging.

As shown in FIG. 1, a preferred exothermically heated elastic adhesive tape 100 includes an exothermic compound layer 10 disposed onto a first of a pair of exothermic compound sealing layers 30, preferably, so that the exothermic compound layer 10 and the exothermic compound sealing layers 30 form a sinusoidal or serpentine path. As shown in FIG. 2, the serpentine pathway is then secured to the first and second thermally insulating elastic fabric layers 50 and 51 by adhesive layers 40, located at least on the centerline of the serpentine pathway, and more specifically the adhesive layers 40 have an adhesive band that engages with the central axis of the serpentine pathway and is preferably applied between the exothermic compound sealing layer 30 and the thermally insulating elastic fabric layers 50 and 51, on each side of the exothermic compound layer 10. Additional portions of the adhesive layers 40 can be located above and below the serpentine pathway, and/or along the transverse ends of the tape 100 or wrap. It is desirable for the serpentine pathway to tend to unwind when the tape 100 is stretched during use, as when it is applied to a wearer. The exothermic compound sealing layers 30 can be broken during use, for example, when they are made of thin polyethylene film, so as to expose the exothermic compound layer 10 to air or oxygen so that the exothermic reaction can begin or resume. In other embodiments, the sealing layer or envelope formed by the exothermic compound sealing layers 30, is made porous to air or oxygen, such as when it contains a porous woven fabric like cotton or rayon, or a polymeric film which has been needled to make it oxygen permeable. In such instances, the packaging for the tape 100 or wrap, can be made of an air or oxygen impermeable polymer film, such as a polypropylene or polyethylene-containing plastic bag, so that when the packaging is removed or torn, the exothermic reaction begins or resumes.

### Sandwich Embodiment:

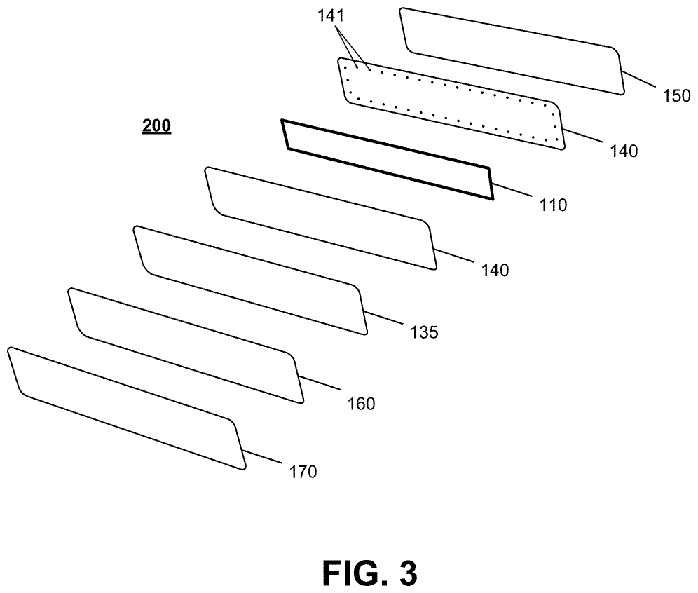

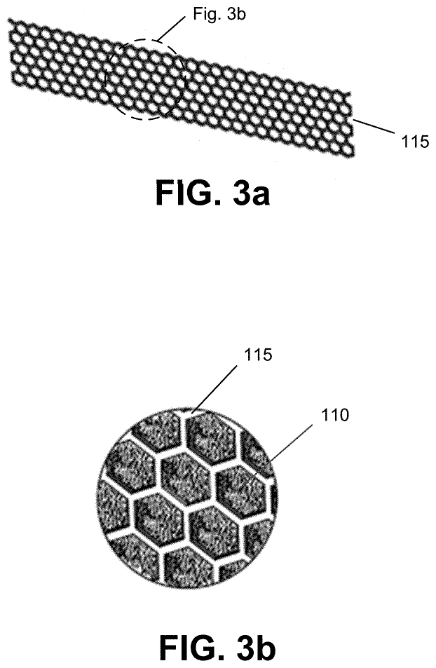

In still a further embodiment of the invention shown in FIGS. 3, 3a, 3b, 4 and 5, an elastic therapeutic wrap or tape 200 is provided, which is adapted for application to the external skin of a wearer. In this tape 200, the exothermic compound layer 110 is disposed between a pair of adhesive layers 140, one of which is optional, and either of which, or both, contain peripheral adhesive 141 (shown) or adhesive disposed over nearly all of the surface areas of the thermally insulating elastic fabric 150 and heat absorbing layer 135, which face one another in the final construction. An thermally insulating elastic fabric 150 is preferably disposed on the top of the tape 200 next to the optional adhesive layer 140, and a heat absorbing fabric layer 135 can be disposed along the bottom adhesive layer 140. A further adhesive layer 160 is used to join the heat absorbing fabric layer 135 to the wearer's skin or clothing. A release liner 170 can be added prior to packaging in a hermetic or oxygen barrier film, such as a polymer film.

Alternatively, the exothermic compound layer 110 can be disposed within (or within and around) the pores of a stretchable polyester mesh fabric 115, as shown in FIGS. 3a and 3b, and more preferably a bag mesh, such as 60″ Heavy weight Polyester Mesh 13 oz./square yard, 100% Polyester mesh or 50″ 8600 Polyester Dive Mesh with pencil sized round holes, both available from Seattle Fabrics, Inc.

https://www.seattlefabrics.com/60-Heavy-Polyester-Mesh-1250-linear-yard_p_81_html

Alternatively, the stretchable polyester mesh fabric 115 can be replaced with a stretchable polyester (or polyester blends) 3D mesh fabric having a thickness of about 2-10 mm and a weight of about 80-600 g/m², such as, for example:

3D Spacer Mesh Fabric Football Pattern—polyester fabric, knitted fabric, home textile mattress; 100% Polyester air mesh fabric; Type Mesh Fabric, Pattern Printed , Style Plain, Width 55/56″ , Technics Knitted, Knitted Type Warp, Waterproof, Flame Retardant, Tear-Resistant, Shrink-Resistant, Yarn Count 100-150d , Weight 386 g/m², Density 386 g/m², Model Number WT504, color black/white or upon request, from TIANRUI TEXTILES CO. LIMITED;

China (mainland) Guangdong Dongguan, No.17, Sanheng Road, Cibian District, Housha Road, Houjie Town, Dongguan City, Guangdong .

### Homepage Address:

www.ttnet.net
Product web page:
https://www.ttnet.net/ttnet/gotoprd/LC150/140/0/13836393838313436313730393433393030343 33939393.htm

Or 100% Polyester 3D Mesh Fabric for Shoes, Car Seat covers and Mattresses; 100% polyester, 75D Pattern (round holes), Model Number: DO-ZL-030; weight: 170 g/m², from Wujiang Do Textile Co. Ltd., Address: No. 22 and 24, Building 16, Wenzhou, Business District, East Silk Market, Shengze Town, Wujiang, Jiangsu, China (215228).

### Homepage Address:

http://www. globalsources.com/dotextile.co

Or 100% Polyester Air Mesh Fabric for Bags, (diamond shaped holes); Model Number: BMDE-030, Weight 120 g/m²; from Wujiang Benmore Textile Imp and Exp Co.,Ltd; Address: Room 417, No. 1 Building, Jinbaisheng Square, Shengze, Wujiang, Jiangsu, China (215228).

### Homepage Address:

http://www.globalsources.com/benmoreterx.co

Or 3D Mesh fabric, 3D Mesh-Color Black, Material: Spandex 30%+Nylon 50%+ Polyester 20%; Thickness: about 3.7 mm; Weight: 500 g/m²; from Danking Enterprise Ltd., Address: 10E-9, No. 374 Bade Road, Sec. 2, Sung Shan District, Taipei, Taiwan (105);

### Homepage Address:

http://www.globalsources.com/danking.co

### Shingle Embodiment:

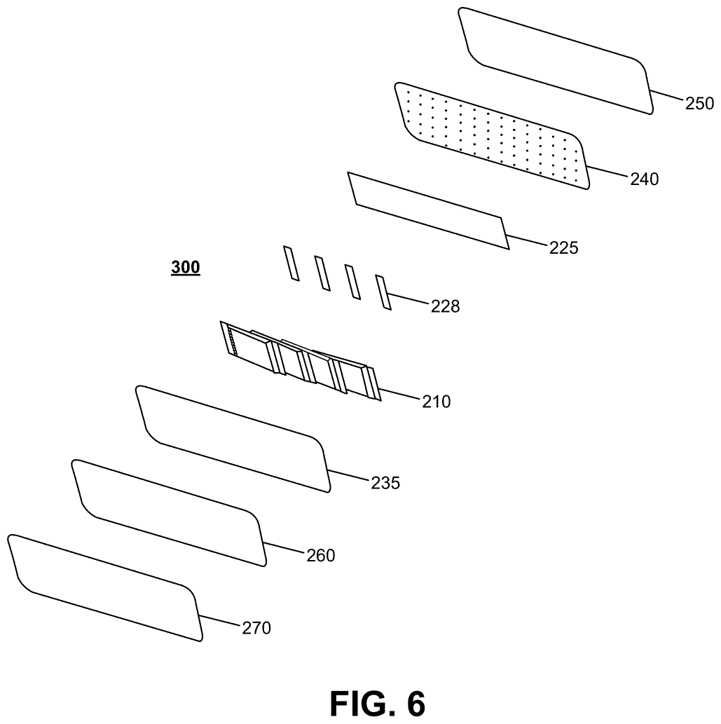

In a further embodiment of the invention shown in FIGS. 6, 7, 8, 9 and 10, an elastic therapeutic wrap or tape 300 is provided, which is adapted for application to the external skin of a wearer. The tape 300 or wrap comprises: an exothermic heating compound 210 disposed within a series of individually wrapped exothermic heating packets 220 of about preferably about 3 cm in width, about 10 cm in length and about 1-4 mm, preferably about 2-3 mm in thickness. The packets 220 contain an exothermic compound 210 wrapped in an oxygen permeable fabric bag (like hand warmers, but smaller and thinner). They are individually adhered to a fabric slip layer 225 by the adhesive layer 228, (which can be a continuous layer or a series of adhesive pads), on one transverse end only of each packet 220, so that they flop over and can partially lay upon one another, like fallen dominos. The adhesive layer 240 for the thermally insulating elastic fabric layer 250 preferably only adheres to the back of the fabric slip layer 225 (non-packet facing side), and around the periphery of the thermally insulating elastic fabric layer 235, FIG. 6. This allows the packets 220 to spread apart during the stretching of the tape 300, FIGS. 9 and 10, while still being adhered to the fabric slip layer 225, so that the exothermic heat can be distributed more evenly along the stretched tape 300 for example, as in kinesiology applications.

As shown in FIG. 10, the packets 220 appear to lay flat on the thermally insulating elastic fabric layer 235 after stretching, or can be still partially overlapping or even be separated, and still provide mostly uniform heat to the wearer along the length of the stretched tape 300. In instances where the packets 220 are substantially separated, the thermally insulating elastic fabric layer 235 can be made thinner or a heat conducting layer could be used instead or in combination, to allow more uniform heat transfer. The fabric slip layer 225 is shown having only intermittent adhesive pads 228 for bonding to the packets 220 of the exothermic heating compound 210, and is preferably not adhesively bonded to the thermally insulating elastic fabric layer 235, so that the fabric slip layer 225 can slide or “slip” over the thermally insulating elastic fabric layer 235 and allow the packets 220 of the exothermic heating layer 210 to move relatively freely and expand the distance between adhesive pads 228 as the tape 300 is stretched.

A further adhesive layer 260 is used to join the thermally insulating elastic fabric layer 235 to the wearer's skin or clothing. A release liner 270 can be added prior to packaging in a hermetic or oxygen barrier film, such as a polymer film.

### Compartmented (Pocketed) Embodiment:

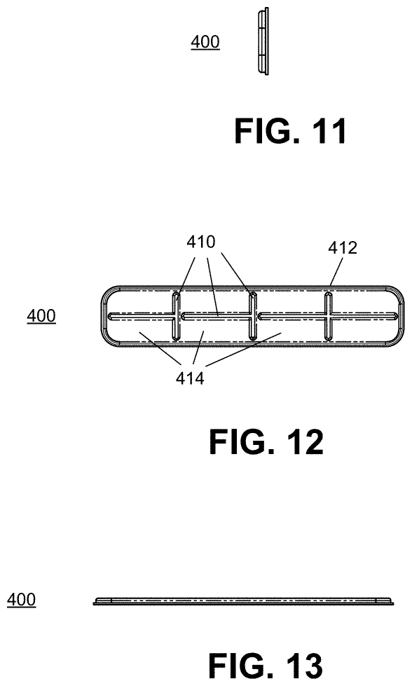

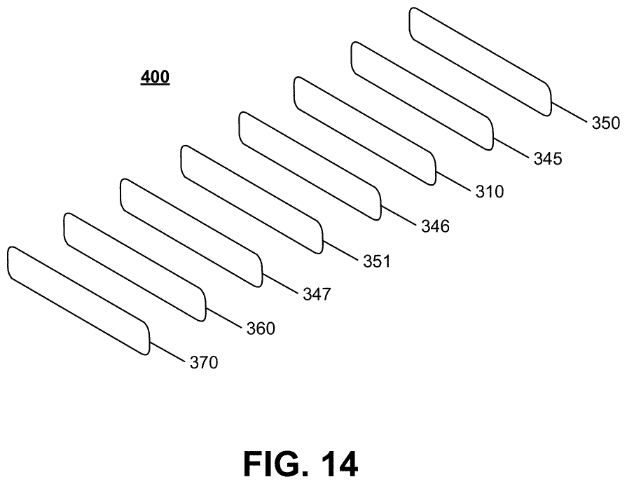

A further embodiment of a preferred tape 400 or wrap of this invention is shown in FIGS. 11-16. The tape 400 or wrap is designed to conform to a portion of the external skin of a body of a wearer. The tape 400 or wrap has a longitudinal length, a width and at least two transverse ends. Preferably, the tape 400 includes first and second elongated elastic layers 345, 346 comprising an elastic knit, weave or film, such as a preferred thermoplastic polyurethane (“TPU”) film having a thickness of no greater than about 0.015-0.02 mm. The first and second elastic layers 345, 346 comprise a peripheral bond 412 and a plurality of intermediate bonds 410. These bonds 412 and 410 join the first and second elastic layers 345, 346 together between the transverse ends of the tape of wrap 400, as shown in FIGS. 12 and 14. An exothermic compound 310 or material is sandwiched between the first and second elastic layers 345 and 346 and is disposed at least between the intermediate bonds 410 or intermediate movement blocking surfaces to form a plurality of compartments or pockets 414 containing exothermic compound 310 disposed along the length of the tape 400 or wrap. The peripheral 412 and/or intermediate bonds 410 preferably form intermediate movement blocking surfaces between the first and second elongated elastic layers 345, 346 so as to help impede the movement of said exothermic material during use. Or, stated another way, the exothermic material 310 can be at least partially bonded, adhered, mechanically secured, partially secured or impeded, or secured by a combination of these methods, within a plurality of pockets or compartments 414 formed between said first and second elongated elastic layers 345, 346, so as to resist accumulating or clumping said exothermic material 310 at one or both ends of said tape 400 or wrap during use. These intermediate movement blocking surfaces can be raised surfaces located on one or both of the first and second elongated elastic layers 345, 346, such as by adding material in the form of ridges, or molding or pressure forming ridges in these layers, or by partially bonding or adhered these surfaces together, by adhesive or melt bonding, the latter including, for example, heat, sonic welding, radio frequency (RF) welding or the like, or they can be grids or mesh layers disposed between the first and second elongated elastic layers 345, 346, like mesh layers 115 in FIG. 3a and b. These intermediate movement blocking surfaces may form a serpentine path between the first and second elongated elastic layers 345, 346 of the tape 400 or wrap, similar to the shape disclosed in FIG. 1.

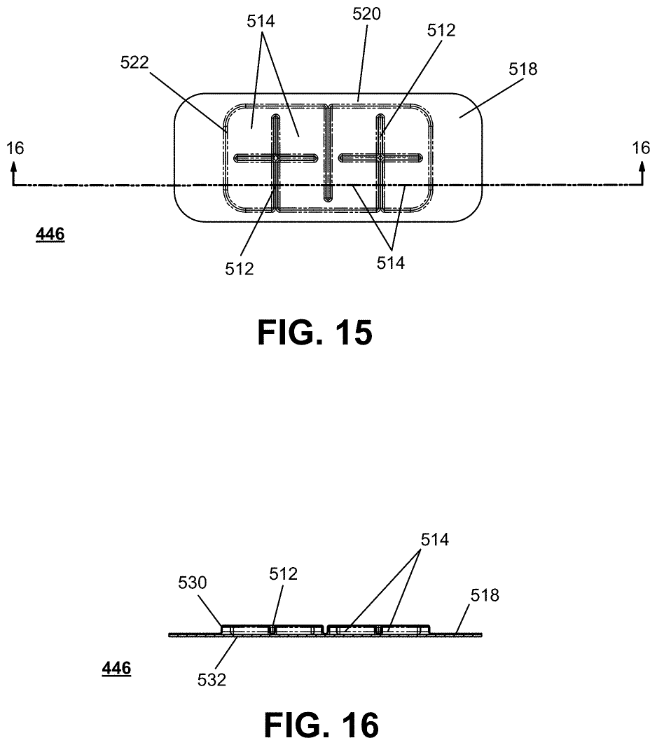

In still a further embodiment, shown in FIGS. 15 and 16, one or both of the first and second elongated elastic layers 345, 346 can be formed, e.g. by thermoforming or vacuum forming or both, to make the first formed elongated elastic layer 530 (top of FIG. 16) having a series of trays 514 formed therein which are preferably defined by ridges 512 formed in the material. In the preferred embodiment, the first formed elongated elastic layer 530 is used for the lower elongated elastic layer, and contains the exothermic material 310. The first formed elongated elastic layer 530 and a second elongated elastic layer 532 (shown upside down) are bonded together to form the elastic exothermic containment envelope 446, see FIG. 16. Layers 530, 532 employ the same or similar materials, such as TPU, as was disclosed for the first and second elongated elastic layers 345, 346 of tape 400, FIG. 14. The elastic exothermic containment envelope 446 also may contain a peripheral seal 522, for sealing around the trays 514 or pockets, after the exothermic material 310, is loaded into the trays 514. The elastic exothermic containment envelope 446 may also contain an anchor lay down region flange 518, which can be longer in the warp direction than the width of the peripheral seal flange 520 is in the weft direction, for allowing the tape to lay down, nice and tight against the wearer's skin. The anchor lay down region flange 518 and the peripheral seal flange 520 are preferably the product of heat sealing layers 530, 532 together. The trays 514 preferably have a depth of about 1/16″ to about ⅛″ and are partially formed by the intermediate movement blocking surfaces (e.g. ridges 512) in both the weft and warp directions. The intermediate movement blocking surfaces can allow partial movement of exothermic material around the small gaps between the bonds and seals, or no movement at all.

In more preferred embodiments, the elastic exothermic containment envelope 446 containing the exothermic material 310, is substituted for the first and second elongated elastic layers 345, 346 in FIG. 14, and the tape 400 is thereafter assembled in a similar manner, preferably employing heat sealing.

In other embodiments, a first laminate is provided containing a first thermally insulating elastic fabric layer made of a knit, weave, woven, or film, or blend, such as 50% nylon/50% polyester blend, or knitted or woven cotton, for example. The elastic fabric layer of the first laminate is preferably laminated to a TPU layer, by heat, adhesive or both. The first laminate is then thermoformed into the same shape as the first formed elongated elastic layer 530 (above, FIG. 16) and filled with particulate exothermic material to a depth of about ⅙″ to ⅛″. A second laminate is then provided which preferably contains a similar elastic fabric, this time, laminated between a pair of TPU layers, one on each side. The process next includes heat sealing the second laminate over the first laminate and its filled trays (like the elastic exothermic containment envelope 446 was sealed in FIGS. 15 and 16), whereby a peripheral heat seal and a plurality of intermediate bonds are formed between the first TPU layer of the second laminate and the TPU ridges and flanges of the filled TPU side of the first laminate. This construction can be further improved by adding a third laminate containing an elastic fabric disposed between a skin adhesive layer (with or without a laminated TPU carrier layer) having a release paper liner over the adhesive, and a further TPU layer. The TPU-elastic fabric-adhesive layer laminate can be heat sealed to the rest of the tape, during the heat sealing process used for joining the first and second laminates together, or in a separate heat sealing step. After the final heat sealing step the final assembly is die cut to final shape.

Referring again to FIG. 14, The first and second elastic layers 345, 346 can be adhesively or melt bonded, for example, with heat, sonic welding, radio frequency (RF) welding, but if carbon is used in the exothermic compound, the compound should be injected between said first and second elastic layers 345, 346 (such as providing the elastic layers 345, 346 in a tube form, such as a TPU tube formed by extrusion, molding or by RF welding a pair of layers together, or by ultrasonic welding, adhesive tape or adhesive spray bonding.

TPU film is preferably bonded using RF or ultrasonic welding or heat bonding. The introduction of exothermic material 310 between the first and second elastic layers 345, 346 can be conducted in an inert environment such as argon gas, or in a vacuum, for example, or in air if the process is conducted quickly enough, so as to preserve the reaction time of the exothermic material. The intermediate bonds 410 or intermediate movement blocking surfaces are positioned to at least partially impede the movement of the exothermic material 310 when the tape or wrap 400 is worn by a user, such as during exercise or when the tape or wrap is stretched and applied to said user.

The exothermic material 310 can be activated by exposing it to oxygen either by opening the package, and exposing a ventilated or oxygen permeable layer, or by breaking a friable layer proximate to the exothermic material 310. Thermally insulating elastic fabric layers 350 and 351 can be made of knits, weaves, wovens, or films, such as 50% nylon/50% polyester blends, or knitted or woven cotton, for example. An additional elongated elastic layer 347, also preferably TPU, can be heat or sonically bonded to the lower thermally insulating elastic fabric layer 351 prior to applying adhesive layer 360 and its release liner 370. Optionally, the adhesive layer 360 can be applied directly to second elongated elastic layer 346 prior to applying the release liner 370.

In a preferred embodiment, the intermediate bonds 410 form a series of square or rectangular pockets 414. The intermediate bonds 410 can be contiguous with the peripheral bond 412, or stop just short of joining to the peripheral bond 412, as shown. Additionally, the intermediate bonds 410 can be separated from one another as shown in FIG. 12, to further encourage stretchability in the tape or wrap 400.

As shown in FIG. 12, the tape 400 or wrap contains two T-shaped intermediate bonds and a cross-shaped intermediate bond, but this is for exemplary purposes only, and there can be multiple configurations (bonds or seals containing dots, dotted lines, curves and circles or ovals) which both maintain stretchability of the first and second elastic layers 345 and 346, while keeping the exothermic material 310 from sliding longitudinally or laterally when the wrap 400 or tape is applied and used.

In a more preferred embodiments, the tape 400 or wrap can be stretched greater than its normal (unused) length and then adhesively applied to the skin of a wearer, so that it will recoil and create a pulling force on the skin. Preferably, this stretchability allows the tape 400 or wrap to stretch up to about 10-90% of its original length, more preferably, up to about 20-70% of its original length.

In a further preferred embodiment, the first and second elastic layers 345 and 346 are prepared in different manners. The second or bottom elastic layer 346 is preferably about 0.15 mm in thickness, which for some TPU films, is breathable to oxygen and water vapor, but is generally water resistant.

One form of commercial TPU useful for this invention is Breathable Polyurethane Film by American Polyfilm, Inc. 15 Baldwin Dr. Branford, Conn.. https://www.americanpolyfilm.com/breathable-tpu-film.

Preferably, the TPU film, coating (such as a laminate including another fabric) or layer is provided to us in unsupported film on rolls. From this form, the TPU sheet film can have adhesive and liner applied or can be die cut to specification. The preferred TPU films perform well in laminations to produce breathable textiles. These TPU films can be provided in 1 mil or greater thicknesses, preferably about 0.015 to about 0.025 mm and in widths up to 78 inches. The preferred films should have high durability, abrasion resistance, and low temperature flexibility.

Preferred films for this purpose, such as TPU or other elastic films, can be monolithic, but create a liquid barrier. They can provide a distinct advantage over other breathable products that are microporous, which means these other products have tiny holes in the film for allowing moisture vapor through. Nevertheless, the preferred films can be breathable without perforation, or can be perforated to provide even greater air or oxygen permeability.

For example, the first or top elastic layer 345 initially can have substantially the same thickness and properties, as the bottom elastic layer 346, but is then perforated with the perforation roll. The perforation step can be performed by an array of from about 10 to about 60 pins/cm2, with, e.g., tapered points and diameters of from about 0.2 mm to about 2 mm, preferably from about 0.4 mm to about 0.9 mm, and more preferably, about 10-14 hole/sq. cm with a size of about 40-60 microns. The perforations allow more oxygen to enter the exothermic compound 310 to initiate and regulate the exothermic reaction. The more holes there are, the hotter the reaction will be and the shorter its duration. Additionally, the greater the size of the holes, the shorter the exothermic reaction will be and it will also be hotter. Conversely, if the number of pins is reduced, or the size of the holes is reduced, the reaction can be longer, and the temperature can be reduced. Thus, the temperature and duration of the exothermic reaction can be calibrated during the construction of the wrap or tape 400.

### Preferred Thermally Insulating Elastic Fabric:

A stretchable, flexible fabric that will contour well to the body.

Insulating (to prevent burning or irritation to skin).

Breathable (to oxygen).

Water repellent or waterproof.

### Preferred Exothermic Compound Sealing Layers and Elongated Elastic Layers:

The preferred exothermic compound sealing layers 30 and elongated elastic layers 345, 346, can be coated fabrics or non-woven films that remain breathable, while securely retaining the exothermic compound, and help to resist initiating the exothermic reaction. The sealing layers 30, elongated elastic layers 345, 346, can provide an “envelope” or “tube”, or formed tray or pocket, which can be elastic and friable or inelastic and friable, or elastic and breathable, but made more oxygen or air permeable on one or both sides.

### Preferred Exothermic Compound:

Disposable heated tapes and wraps of this invention preferably employ a one-time exothermic chemical reaction generated by an exothermic compound 10, 110, 210, 310. One type, frequently used for hand warmers, can be activated by unwrapping an airtight packet containing slightly moist iron powder and salt, or a catalyst, which can rust over a period of hours after being exposed to oxygen in the air. See https://en.wikipedia.org/wiki/Heating.pad. Another type contains separate compartments; when the user squeezes the wrap or tape, a barrier ruptures and the compartments mix, producing heat such as the enthalpy change of solution of calcium chloride dissolving.

The wraps or tape can also contain a supersaturated solution of sodium acetate in water. Crystallization is triggered by flexing a small flat disc of notched ferrous metal embedded in the liquid. Pressing the disc releases very tiny adhered crystals of sodium acetate into the solution which then act as nucleation sites for the crystallization of the sodium acetate into the hydrated salt (sodium acetate trihydrate, CH3COONa.3H20). Because the liquid is supersaturated, this makes the solution crystallize suddenly, thereby releasing the energy of the crystal lattice.

The sodium acetate—containing wraps or tapes can be reused by placing them in boiling water for 10-15 minutes, which redissolves the sodium acetate trihydrate in the contained water and recreates a supersaturated solution. Once the wrap or tape has returned to room temperature it can be triggered again. Triggering the wrap or tape before it has reached room temperature results in the pad reaching a lower peak temperature, as compared to waiting until it had completely cooled.

The preferred exothermic compound 10, 110, 210, 310 can be made to achieve a specific target temperature and heating duration , such as about 100-135 F (about 40-70 C), and more preferably about 122 F+/−10 F, 50 C+/−5 C for 30 min.-6 hours, from environmentally safe materials such as iron powder, water, salt, activated charcoal & vermiculite. The exothermic compound 10, 110, 210, 310 is preferably disposed within fabric layers which allow oxygen to activate the compound, but keep the compound particles from leaking out, while allowing heat to flow at least in the direction of the wearer's skin, while also allowing 10-50% stretch in the tape as applied. The exothermic compound 10, 110, 210, 310 is preferably single use and can be disposed safely.

### Preferred Adhesives:

The adhesive layers, 40, 60, 140, 160, 240, 228, 260, 360 (or at least the adhesive layers intended to face the skin) should be skin friendly—tested for and meets the ISO 10993 standards for skin sensitization and irritation.
Latex free, such as acrylic or silicon based adhesives
Hypoallergenic or skin friendly, tested to meet ISO 10993 standards for skin sensitization, irritation and cytotoxcicity.
Moisture friendly—maintains a high Moisture Vapor Transmission Rate (MVTR).
Breathable—for adhesive and skin stability.
Stretchable—to accommodate the elastic characteristics of the adhesive tape.
Repositionable—may be repositioned to the skin with a low-peel release force that does not cause damage or pain, yet maintains tack and adhesion after several applications.

The adhesive layers 40, 60, 160, 140, 240, 228, 260, 360 can be two-sided adhesive tape or sprayed or roll applied adhesive layers. The adhesive layers 40, for example, are used to adhere one or both of the preferred elastic fabric insulating layers 50, such as upper and lower layers mentioned above in connection with the preferred embodiment 100. An adhesive layer 60 is provided for contact with the wearer's skin or clothing, and a release liner layer 70 is applied over the adhesive layer 50.

### Fabric and Film Selection

Nonwovens, and woven fabrics (including films), if used, provide support and integrity to the exothermic compounds. Examples of suitable films include polyethylene, polypropylene, nylon, polyester, TPE, polyvinyl chloride, polyvinylidene chloride, polyurethane, polystyrene, saponified ethylene-vinyl acetate copolymer, ethylene-vinyl acetate copolymer, natural rubber, reclaimed rubber, and synthetic rubber. The exothermic compound sealing layers 30 thickness is preferably in the range of about 1 to about 300 μm and may be oxygen permeable or impermeable, or have areas that are selectively oxygen permeable or impermeable.

Fabrics useful in embodiments 100, 200, 300 and 400 should have preferred characteristic properties of light weight and good tensile strength, e.g., nylon, rayon, cellulose ester, polyvinyl derivatives, polyolefins, polyamides, or polyesters, cuproammonium cellulose (Bemberg) and other high molecular weight compounds, as well as natural materials such as, wool, silk, jute, hemp, cotton, linen, sisal, or ramie, are suitable, as are blends containing some or all of these types of materials.

### Stretch Fabrics

Stretch fabrics are either 2-way stretch or 4-way stretch. 2-way stretch fabrics stretch in one direction, usually from selvedge to selvedge (but can be in other directions depending on the knit). 4-way stretch fabrics, such as spandex, stretches in both directions, crosswise (weft) and lengthwise (warp). Stretchy materials refers to fabrics which can stretch without breaking the fibers and return to its original length. This stretch of the fibers provides the preferred tapes and wraps of this invention made with them the much needed ease, drape, comfort and fitting. See https://sewguide.com/stretchy-fabric, which is incorporated herein by reference.

### Knits:

Most of the knit fabrics have some stretch, even without Spandex or another elastomer. Usually, it is a 2-way stretch, with less stretch in the weft direction. The stretch of a knit fabric makes it one of the most suitable fabric for tape and wraps. Jersey is a light to medium weight knit fabric with good stretch. Other suitable knit fabrics are: 3×3 Rib Knit, Bamboo Jersey, Double knit Rayon Blend, Interlock twist jersey, Double knit, Sweater Knit, Silk Mesh Knits and Silk Jersey.

### Spandex:

Also suitable for this invention are Spandex fabrics, which is a generic name for stretchy fabrics with elastic content—the base could be cotton, nylon or wool and Lycra, a spandex fabric, which is trademarked by Dupont Company. Its ability to stretch to almost 300-400 percentage on its own. The stretchiness of Spandex depends on its elastane content; 1-5% is desirable, whereas for sportswear an elastane content of about 12-15% is more preferred Spandex blends.

### Spandex Blends:

When blended with other fibers spandex can lend about 2-20% of its elasticity to the new fibers, such as cotton wovens.

### Cotton—Spandex Blend

Very smooth fabric with a 4 way stretch. The percentage of spandex usually is 3-5%. It can be used to make tape and wraps.

### Polyester—Spandex Blend

This is very lightweight stretchy material with stretch.

### Stretch Satin

This is a polyester -spandex blend in a satin finish.

### Stretch Velvet

This fabric with a napped surface can have about 15% stretch.

### Polyester Spandex Fabric

Polyester spandex fabric with or without foam backing.

### Nylon Spandex Fabric

This stretchy material can be used for active or sports applications. It has a very nice 4-way stretch (15% spandex mostly). Tricot (Italian Tricot etc.) is a Nylon Spandex which has 25-50% stretch.

Rayon knit/Spandex

This has a good stretch and it is a very silk like smooth. This combination make it ideal for making very fitting and comfort against the skin. Bamboo rayon is especially smooth.

### Acetate/Spandex; Modal/Spandex; Tencel/Spandex; Linen/Spandex

Acetate/Spandex; Modal/Spandex; Tencel/Spandex; Linen/Spandexare other blends which are available.

### Stretch Denim

Stretch Denim is a lightweight denim with a stretch for comfort and ease. It is a mix of cotton, polyester and spandex.

### Cotton Poplin Stretch

Cotton poplin stretch is a stretchy fabric is a blend of Poplin, cotton and spandex—this fabric usually has a slight (10%) stretch and is very comfortable .

### Rubber/Latex:

Latex is made from the sap of the rubber tree. Rubber can be used to make tape and wraps but may not be suitable for extended use. It is has little to no breathing ability and at times may be irritating to skin, but can be perforated. But the stretch of rubber is even more than even spandex and water resistance makes it desirable for certain applications.

### Neoprene Rubber:

Neoprene rubber is a synthetic polymer resembling rubber. Wet suits used in scuba diving are usually made of Neoprene rubber. Neoprene rubber is available in many thicknesses and can be used to make wraps and tape with the heating elements of this invention.

And finally woven fabric can also stretch—when it is cut on the bias. So if you want the advantages of a woven fabric, it can be cut on the bias grain i.e. along a line drawn 45 degrees to the lengthwise and crosswise grain of the fabric, to provide more of a stretch.

### Nonwoven Fabrics and Films:

Nonwoven materials are generally described in Riedel “Nonwoven Bonding Methods and Materials”, Nonwoven World, (1987), incorporated herein by reference in its entirety. An example of a suitable commercially available polypropylene/ethylene vinyl acetate (PP/EVA) film is material number DH245, which is commercially available from Clopay Plastics of Cincinnati, Ohio U.S.A. Another useful material is thermoplastic elastomer film (TPE). Thermoplastic elastomers combine the mechanical properties of rubber-based materials (e.g. high elasticity, abrasion resistance, and friction) with the good processability and recyclability of thermoplastics. This TPE film can be stretched up to 600 percent before it breaks. It is temperature resistant from −50° C. to 100° C. (unstretched material) and has good chemical and UV resistance. It is food safe and does not contain latex (to which some people are allergic). Thermoplastic Polyurethane (TPU) film is another tough, versatile elastomer which is ideal for many applications of this invention. TPU is inherently soft and generally requires no plasticizers. Compared to other polymers, TPU-based films exhibit toughness, flexibility, and abrasion resistance. In addition, these polymers are versatile and can be formulated for low surface tack, improved light stability, and antimicrobial properties.

TPU films can be derived from three different TPU chemistries, all of which can be formed and fabricated by conventional methods and are receptive to Radio Frequency (RF) welding. Generally, ether-based grades resist mold and hydrolysis when exposed to water and high humidity environments, while ester-based grades are generally preferred for applications where the film will be in contact with oils or fuels. For outdoor applications requiring a clear, non-yellowing product, aliphatic TPUs are preferred. All of these grades are available in a range of sizes and surface embossments.

Traditionally, TPU films are a natural/clear color; however, custom color matching and other tailored performance attributes are available upon request. Plasticizers are typically not required as TPU is inherently soft. It is also notable that the softness or hardness of these polymers remains relatively consistent over a wide temperature range for extended periods of time. For further functionality we can laminate TPU and TPU blended films with other films (dual durometer), non-woven fabrics, hook receptive loop fabric, and reinforcing scrims to create new properties. Unsupported TPU grades are available in film thicknesses from 0.006″ to 0.125″ (0.2-3.175 mm). https://www.winmancorp.com/wp-content/uploads/Winman-Thermoplastic-Polyurethane-Film.pdf

Thermoplastic polyurethane on silicone coated release liner is available in thicknesses from 0.003″ to 0.012″ (0.075-0.305 mm). An example of a suitable commercially available TPE is DuPont™ Hytrel® 7246 film. The fabrics and materials mentioned in this paragraph can also be used for the elastic fabric insulating layers 50. See also https://www.wimancorp.com/thermoplastic-films/thermoplastic-polyurethane-blends/thermoplastic-polyurethane-tpu-films/; which is also incorporated herein by reference.

In the most preferred embodiments, selected TPU film layers are both pervious to oxygen (hereinafter “breathable”) and impervious to liquid water (such as the water in the exothermic compound or sweat from the wearer) (hereinafter “waterproof”). TPU film is generally breathable and waterproof in films having thicknesses of about 0.001-1.5 mm, preferably about 0.001-0.05 mm, and more preferably about 0.015 and 0.02 mm; any thicker the material may not breathable, without further treatment, such as perforation. We prefer to use a TPU film having a thickness of about 0.015 mm, in order to maintain the elasticity of the wrap or elastic tape, while also having a layer which is 90%-99% breathable and waterproof. It is also important to note that such TPU films are biocompatible and, more importantly, antimicrobial.

TABLE 1
Properties of TPU films:

| | | | | | | | | | | Aliphatic |
|---|---|---|---|---|---|---|---|---|---|---|
| | | Ether-based Grades | | | | | | Ester-based Grades | | Grade |
| Property | Test Method | Unit | ET-90070 | ET-90083 | ET-50087 | ET-90090 | ET-90092 | ES-20085 | ES-20092 | AT-90090 |
| Specific | ASTM D 792 | — | 1.07 | 1.11 | 1.12 | 1.13 | 1.14 | 1.20 | 1.22 | 1.08 |
| Gravity | | | | | | | | | | |
| Durometer | ASTM D 2240 | — | 72 | 84 | 87 | 90 | 92 | 83 | 93 | 90 |
| (5 second | | | | | | | | | | |
| dwell) | | | | | | | | | | |
| Tensile | ASTM D 882 | psi | 5000 | 5700 | 6300 | 7700 | 8700 | 7300 | 8200 | 7500 |
| Strength | | | | | | | | | | |
| Elongation | ASTM D 882 | % | 850 | 720 | 630 | 680 | 650 | 700 | 480 | 550 |
| @ Break | | | | | | | | | | |
| Stress @ | ASTM D 882 | psi | 485 | 850 | 1100 | 1100 | 1400 | 900 | 1800 | 1500 |
| 100% | | | | | | | | | | |
| Elongation | | | | | | | | | | |
| Stress @ | ASTM D 882 | psi | 700 | 1300 | 2100 | 2300 | 3400 | 2000 | 5000 | 3500 |
| 300% | | | | | | | | | | |
| Elongation | | | | | | | | | | |
| Tear | ASTM D 624 | lbf/in | 350 | 450 | 510 | 500 | 500 | 550 | 700 | 480 |
| Strength | | | | | | | | | | |
| Taber | ASTM D 3489 | mg Loss | 7 | 30 | 40 | 25 | 55 | 35 | 27 | — |
| Abrasion | | | | | | | | | | |
| (H18, 1000 g, | | | | | | | | | | |
| 1000 cycles) | | | | | | | | | | |
| Glass | Internal | F. | −68 | −49 | −49 | −47 | −40 | −44 | 5 | −40 |
| Transition | | | | | | | | | | |
| Temperature | | | | | | | | | | |

Generally, it is preferred the we use a fabric or film which has a:
Tensile Strength of about 2000-10000 psi, preferably about 4500-9570 psi;
Stress at 100% elongation of about 200-3000 psi, preferably about 436-1980 psi;
Tear Strength of about 100-1000 lbf/in, preferably about 315-770 lbf/in; and
Glass Transition Temperature of about −100 F-+10 F, preferably about −75 F-−36 F.

For kinesiology tape, the tape can stretch anisotropically (in some but not all directions), up to 10-90%, preferably about 20-70% in the warp direction and only about 5-20%, preferably about 10% in the weft direction. Or, isotropically (in all directions) about 10-90%, preferably about 20-75% in both the warp and weft directions. For other applications, the fabric layer can be closer to isotropic in stretchability, such as about 10-80% in the warp direction and about 10-80% in the weft direction.

### Preferred Exothermic Compositions:

The heat cells and pads of the present invention preferably comprise particulate exothermic compositions. The particulate exothermic composition provides for improved sustained temperature when the heat cells and pads are incorporated into disposable heating devices to relieve discomfort of temporary or chronic body aches and pains.

The preferred exothermic compound layers (or materials) 10, 110, 210 and 310 of this invention contain compositions that are particulate exothermic compositions, such as those described in U.S. Pat. No. 7,878,187, which is hereby incorporated herein by reference. As used herein “particulate” refers to separate particles contained within the compositions. In other words, the preferred particulate exothermic compositions described below preferably contain separate particles wherein each particle has a median particle size ranging from about 25 μm (microns) to about 800 μm.

Variations in the particle size of the particulate components of the exothermic compositions defined herein can lead to particle separation or segregation within an exothermic composition. In other words, particle size directly effects particle mobility, and the particulate components defined herein can vary in their mobility resulting in particle separation or segregation. The exothermic compositions defined below preferably comprise particulate components having defined median particle size ranges such that the exothermic compositions preferably resist particle separation or segregation. It is contemplated, however, that particulate components having median particle sizes ranges above or below the ranges defined herein are also suitable for use in the exothermic compositions defined herein.

As used herein “sustained temperature” refers to temperatures ranging from about 32° C. to about 70° C. (89.6-158 F), preferably from about 50° C. (122 F), and more preferably about 38° C. to about 46° C. (100-115 F) for a period of time from about twenty seconds to about twenty-four hours, preferably from about twenty minutes to about twenty hours, more preferably from about one hour to about four hours, wherein the maximum skin temperature and the length of time of maintaining the skin temperature at the maximum skin temperature may be appropriately selected by a person needing such treatment such that the desired therapeutic benefits are achieved without any adverse events such as skin burns which may be incurred by using a high temperature for a long period of time.

Maintaining a “sustained temperature” provided by the particulate exothermic compositions of the present invention has been shown to substantially relieve acute, recurrent, and/or chronic pain including skeletal, muscular, and/or referred pain, of a person having such pain, and to substantially prolong relief even after a disposable heating device comprising the particulate exothermic composition is removed from the afflicted body part without any adverse events.

As used herein, the term “disposable” refers to devices that are intended to be thrown away after extended use. In other words, “disposable” heating devices defined herein are those devices that are meant to be deposited in a suitable trash receptacle after the heating device has been used to release the heat provided by the heat cells. The disposable tapes 100, 200, 300 and 400 or wraps can be stored in a resealable, substantially air-impermeable container for repeated use in the relief of temporary or chronic body aches and pain until the disposable heating device has been fully extended in the release of heat.

All disclosed percentages, parts and ratios are by weight of the particulate exothermic compositions, unless otherwise specified. All such weights as they pertain to listed ingredients are based on the specific ingredient level and, therefore, do not include carriers or by-products that may be included in commercially available materials, unless otherwise specified.

### A Preferred Heat Area or Pathway

The present invention can be directed to heat areas or pathways containing cells, compartments, impediments to movement of exothermic material, mesh cells, pockets or pathways that comprise a exothermic composition. The heat cells or pathways can be incorporated into disposable heating devices to provide for improved sustained temperature in the relief of temporary or chronic body aches and pain. The preferred heat cells are incorporated into the disposable heating devices as a single continuous path or a plurality of heat cell regions or areas (hereinafter “heat pathway” or “heated area”).

The heated area or heat pathway is formed in, or contained within, a unified structure comprising at least two opposed surfaces, wherein at least one surface, or both, is/are oxygen permeable, or made to be oxygen permeable by manual manipulation, such as by stretching or squeezing the product, for example. The volume of the heated area or heat pathway can be filled with a particulate exothermic composition, which has a fill volume, void volume, and a cell volume. The fill volume, as used herein, means the volume of the particulate composition in the filled heated area or heat pathway. The void volume, as used herein, means the volume of the area or pathway left unfilled by the particulate composition in a finished heated area or heat pathway, measured without differential pressure in the heated area or heat pathway and without additional stretching or deformation of the substrate material. The cell or pathway volume, as used herein, means the fill volume plus the void volume. The ratio of fill volume to heated area or heat pathway volume is from about 0.7 to about 1.0, preferably from about 0.75 to about 1.0, more preferably from about 0.8 to about 1.0, even more preferably from about 0.85 to about 1.0, and most preferably from about 0.9 to about 1.0.

As previously stated, the heated area or heat pathway is formed in a unified structure comprising at least two opposed surfaces, which can be film or fabric layers disposed around the exothermic compound layer, such as sealing layers 30 or first and second elongated elastic layers 345, 346. The heated area or heat pathway can be a continuous form like a serpentine path or a series of pockets or cells in a shingle formation or a thin strip as in the sandwich formation. The exothermic compound sealing layers 30 or elongated elastic layers 345, 346 that form the tube or heated area are preferably made of films, films laminated or adhered to nonwoven or woven fabrics or simply nonwoven or woven fabrics.

Although preferred constructions of tapes 100, 200, 300 and 400 and other variations of the invention disclosed, suggest adhesive bonding, alternative bonding methods can be used in almost every place adhesive bonding is disclosed herein. In general the preferred films and fabrics are those having heat sealability and are capable of being easily thermally fused, or can be adhered with, for example, ultrasonic welding, impulse bonding, radio frequency bonding (less desirable due to carbon arcing in the exothermic compound), heat sealing and/or adhesives, hot melt glue, pressure sensitive adhesive or two sided adhesive tape. In certain embodiments, a 0.015 mm layer of TPU is laminated by adhesive and a heated roll to a stretch fabric, such as rayon, nylon, polyester, cotton woven fabrics, or an elastic knit fabric made from these materials.

If the enclosure for the heat pathway or heated area is to be exposed to oxygen during manual manipulation, such as the exothermic compound sealing layers 30, or first and second elastic layers 345, 346 should have air holes or perforations, or should have a relatively low tensile strength, or have thinned or scored areas which enable easier tearing or breaking. Films containing LDPE/LLDPE , HDPE ,PP, PVC, PET (such as Straight Tear PET Films) and other resins and blends, such as PP having a PVDC coating or acrylic, (to make them more oxygen impermeable) are useful for this purpose. Alternatively, a thicker film or a film having a higher tensile strength may be used if an area of oxygen permeability is temporarily sealed, such as by forming a seal over the area of oxygen permeability by heat or glue, and then opening the seal manually before use, such as would be the case if a pin perforated area of the film is hidden and sealed from the environment by a folded portion of the film, so that the seal can be broken when the tape 100 is manually manipulated, such as by stretching to expose the area containing the pin holes. Alternatively, the sealing layers 30 could be adhered (via adhesive, sonic or heat bonding) intermittently to the elastic fabric insulating layers 50, so that when the elastic fabric insulating layers 50 are stretched, the adhered locations grip and tear the exothermic compound sealing layers 30 and expose the exothermic compound layer 10 to oxygen. Finally the sealing layers 30 and

The opposed surfaces of the exothermic compound sealing layers 30 and elongated elastic layers 345, 346 can be created by bonding two substrates together around their periphery to form a pouch, envelope, or pocket or by using a tube, such as an extruded tube. Pockets can also be made in the substrates by thermoforming, mechanical embossing, vacuum embossing, or other acceptable means.

The oxygen permeability of the exothermic compound sealing layers 30 and elongated elastic layers 345, 346 of the present invention can be provided by selecting films or film coatings for the film layer substrates for forming the pouches, impediment areas, envelopes, pockets, and/or covering layer, that have the specifically desired permeability properties. The desired permeability properties may be provided by inherently porous materials, microporous films or by films which have pores or holes formed therein. The formation of these holes/pores may be via extrusion cast/vacuum formation or by hot needle aperturing.

Oxygen permeability can also be provided in the present invention by perforating at least one of the exothermic compound sealing layers 30 or elongated elastic layers 345, 346 with aeration holes using, for example, at least one needle or pin, preferably an array of from about 10 to about 60 pins, with, e.g., tapered points and diameters of from about 0.002 mm to about 2 mm, preferably from about 0.4 mm to about 0.9 mm.

Alternatively, after the exothermic compound sealing layers 30 and elongated elastic layers 345, 346 have been bonded together, to enclose the exothermic compound layer 10, 310 in the pockets between them, one side may be perforated with aeration holes. Equipment useful in making such holes is supplied by Burckhardt of Switzerland AG, Pfarrgasse 11, CH-4019 Basel Switzerland, see “Cold Pin Perforating Unit KPF”. (Available Hole sizes—0<0.02 mm-ca. 3 mm (depending on requirement, material and density of pins); pin density up to 303 pins/cm2 when working with segments (no space between the rings), single rings with higher density possible; high concentricity; pin projection from 0.3 mm-ca. 12 mm; almost any pin and hole arrangement is possible, linear, spiral—patterns available).

The pins are pressed through one side of the heat pathway to a depth of from about 2% to about 100%, preferably from about 20% to about 100%, and more preferably from about 50% to about 100% into the exothermic compound layer 10. This hole configuration provides an oxygen diffusion into the heat pathway during oxidation of the preferred particulate exothermic composition of from about 0.01 cc O2/min./5 cm2 to about 15.0 cc O2/min./5 cm2 (at 21° C., 1 ATM), preferably from about 0.9 cc O2/min./5 cm2 to about 3 cc O2/min./5 cm2 (at 21° C., 1 ATM).

Although there are preferably provided aeration holes in the upper one of the exothermic compound sealing layers 30 or elongated elastic layers 345, 346, it is also possible to provide aeration holes in the lower one of the exothermic compound sealing layers 30 or elongated elastic layers 345, 346, or in all layers.

The exothermic compound layer 10, 110 and 210 or other layer in the tape 100 of the present invention may optionally incorporate a component to be delivered through the skin, wherein the optional component includes active aromatic compounds, non-active aromatic compounds, pharmaceutical actives or other therapeutic agents, and mixtures thereof. The optional component can be incorporated into the tape 100, 200 or 300 or wrap as a separate substrate layer or incorporated into at least one of the exothermic compound sealing layers 30, pads or adhesive layers 40, 60, 160, 140, 240, 228, 260, or in the elastic fabric insulating layers 50, 150 and 250. Such active aromatic compounds include, but are not limited to, menthol, camphor, eucalyptus, and mixtures thereof. Such non-active aromatic compounds include, but are not limited to, benzaldehyde, citral, decanal, aldehyde, and mixtures thereof. Such pharmaceutical actives/therapeutic agents include, but are not limited to antibiotics, vitamins, antiviral agents, analgesics, anti-inflammatory agents, antipruritics, antipyretics, anesthetic agents, antifungals, antimicrobials, and mixtures thereof. The tape 100 or wrap may also comprise a sweat-absorbing component or deodorant.

### Exothermic Composition

The exothermic compound layer 10, 100 and 210 of the present invention preferably comprises a particulate exothermic composition (herein defined as a composition which generates a chemical reaction that releases energy through light or heat, such as a compound that generates heat when exposed to oxygen, for example) which provides for improved sustained temperature when the exothermic compound layer 10 is incorporated into disposable heating devices such as disposable tapes and body wraps. The particulate exothermic composition preferably comprises a particulate premix composition and a brine solution.

Components of the particulate premix composition typically include iron powder, carbon, absorbent gelling material, and water, which components are described in detail hereinafter. Likewise, typical components of the brine solution include a metal salt, water, and optionally a hydrogen gas inhibitor such as sodium thiosulfate. The exothermic compositions defined herein are generally prepared by constructing the particulate pre-mix composition and rapidly dosing the pre-mix with the brine solution to result in the formation of heat cells of the present invention. A typical heat cell of the present invention can comprise from about 0.4 grams of premix per heated area or heat pathway to about 2.5-10 grams of premix per heated area or heat pathway, and from about 0.4 grams of brine solution per heated area or heat pathway to about 1.5-8 grams of brine solution per heated area or heat pathway. Therefore, an exothermic composition of the present invention can comprise a total cell weight, per cell or small region, of from about 0.8 grams to about 18.0 grams, preferably from about 1.5 grams to about 10.0 grams.

The velocity, duration, and temperature of the thermogenic oxidation reaction of the particulate exothermic composition can be controlled as desired by changing the area of contact with air, more specifically, by changing the oxygen diffusion/permeability. Other methods of modifying the exothermic reaction include choice of components within the composition, for example, by choosing a specific component described hereinafter, modifying component particle size, and so forth.

By way of illustration, one particular method of modifying the exothermic reaction involves adding iron powder having a median particle size of about 200 μm, and an absorbent gelling material having a median particle size of about 300 μm, wherein the median particle size ratio of absorbent gelling material to iron powder is 1.5:1. This select ratio of absorbent gelling material to iron powder can provide for an exothermic composition that exhibits a fast initial heating temperature and a long duration of heat, which has been a difficult accomplishment of current exothermic compositions. It is believed that some exothermic compositions comprise a high level of moisture that results in water in the interstitial particle voids, which restricts oxygen flow and slows up the rate of the initial heating temperature. It has been found that exothermic compositions which comprise a select median particle size ratio of absorbent gelling material to iron powder provides for excess water being vacant from interstitial particle voids such that faster rates of initial heating temperatures are achieved.

### Iron Powder

The particulate exothermic compositions of the present invention comprise one or more iron powder components at concentrations ranging from about 10% to about 90%, preferably from about 30% to about 88%, more preferably from about 50% to about 87%, by weight of the composition.

It is believed that the particulate exothermic compositions defined herein release heat upon oxidation of the iron powder. It is known that iron is the anode for the electrochemical reaction involved in the exothermic oxidation of iron. There is no particular limitation to the purity, kind, size, etc., of the iron powder as long as it can be used to produce heat-generation with electrically conducting water and air. For example, iron powder having a median particle size of from about 50 μm to about 400 μm, preferably from about 100 μm to about 400 μm, more preferably from about 150 μm to about 300 μm, have been found suitable for use herein.

The median particle size of the iron powder, and any other particulate component defined herein, can be determined using a sieve method such as the method disclosed in ASTM Method B214.

Preferably, the particulate exothermic compositions comprise a select median particle size ratio of absorbent gelling material defined hereinbelow and the iron powder. Exothermic compositions comprising this select median particle size ratio of components have been shown to provide for heat cells that have improved heat application and that have the ability to resist compositional changes such as resistance to particle segregation. The median particle size ratio of absorbent gelling material to iron powder typically ranges from about 10:1 to about 1:10, preferably from about 7:1 to about 1:7, more preferably from about 5:1 to about 1:5, and most preferably from about 3:1 to about 1:3.

The tapes and wraps of the present invention are typically much thinner as compared to current hand warmers, and excess levels of exothermic composition cannot be used to compensate for particle segregation effects. In fact, adding excess levels of exothermic composition can result in significant changes in the thermal performance of heat cells. It has been found that particle segregation effects are reduced by using iron powder having a median particle size within the ranges defined herein, especially by using iron powder in a ratio combination of absorbent gelling material to the iron powder. It is believed that the reaction rate of exothermic compositions is controlled by the porosity of the exothermic compositions, in other words the rate at which heat cells emit heat is impacted by the packing behavior of the particles (i.e., interstitial particle void volume) and by the amount of water present in the exothermic composition. The iron powder defined herein provides for low packing behavior, whereas the absorbent gelling material prevents water from entering particle voids, thus resulting in heat cells that exhibit fast initial heating temperatures and long duration of heat for treating temporary or chronic body aches and pain.

Non-limiting examples of suitable sources for the iron powder of the present invention include cast iron powder, reduced iron powder, electrolytic iron powder, scrap iron powder, sponge iron, pig iron, wrought iron, various steels, iron alloys, treated varieties of these iron sources, and mixtures thereof. Sponge iron is preferred.

Sponge iron is one source of the iron powder, which may be particularly advantageous due to the high internal surface area of sponge iron. As the internal surface area is orders of magnitude greater than the external surface area, reactivity may not be controlled by particle size. Nonlimiting examples of commercially available sponge iron include M-100 and F-417, which are available from the Hoeganaes Corporation located in New Jersey, U.S.A.

Sponge iron is a material utilized in the steel making industry as a basic source for the production of steel. Without intending to be limited by any method of production, sponge iron may be produced by exposing hematite (Fe2O3) iron ore in comminuted form to a reducing gas environment at temperatures somewhat below blast furnace temperatures.

While oxygen is necessary for the oxidation reaction of iron to occur, an internal oxygen source is not required in the heat cells of the present invention, however, oxygen-producing chemical materials may be incorporated in the particulate exothermic composition at the time of preparation thereof without changing the scope of the present invention. The oxygen sources used for the purpose of this invention include air and artificially made oxygen of various purity. Among these oxygen sources, air is preferred since it is the most convenient and inexpensive.

### Carbon

The particulate exothermic compositions of the present invention comprise one or more carbon components at concentrations ranging from about 1% to about 25%, preferably from about 1% to about 15%, more preferably from about 1% to about 10%, by weight of the composition.

Nonlimiting examples of carbon suitable for use herein include activated carbon, non-activated carbon, and mixtures thereof. The carbon component has a median particle size of from about 25 μm to about 200 μm, preferably from about 50 μm to about 100 μm. Activated carbon is preferred.

Activated carbon serves as the cathode for the electrochemical reaction involved in the exothermic oxidation of iron. However, the cathode capabilities can be extended by additionally using non-activated carbon powder, i.e., carbon blended to reduce cost. Therefore, mixtures of the above carbons are useful in the present invention as well.

Activated carbon is extremely porous in the inner structure giving it particularly good oxygen adsorption capabilities. In fact, activated carbon has the ability to adsorb oxygen extremely well when the activated carbon is wetted, thus allowing for the activated carbon to function as a catalyst in the electrochemical reaction.

Moreover, activated carbon can absorb water well, and can serve as a water-holding material. Further, active carbon can adsorb odors such as those caused by the oxidation of iron powder.

To provide for fast heat up of the exothermic composition while sustaining thermal duration, the exothermic compositions can optionally have more absorbent gelling material than the activated carbon. It has been shown that if the absorbent gelling material is less than the activated carbon, then the exothermic reaction becomes sensitive to the moisture content and will not heat up as fast.

Additionally, the amount of carbon in the particulate exothermic compositions defined herein should be minimal in order to maximize the interstitial particle void volume. Carbon is typically the finest particle component and excess carbon would result in the carbon filling up the interstitial particle void volume.

A low level of carbon is also highly desirable for the method of making heat pathways and areas of the present invention since a low level of carbon provides for the pre-mix to rapidly absorb the brine solution. This significantly increases the rate of the method of making the heat pathways and areas defined herein.

### Optional Absorbent Gelling Material

The particulate exothermic compositions of the present invention optionally include one or more absorbent gelling materials at concentrations ranging from about 1% to about 25%, preferably from about 1% to about 15%, more preferably from about 1% to about 10%, by weight of the composition.

The absorbent gelling material suitable for use herein enables the retention of water physically or chemically within the particulate exothermic compositions of the present invention. In particular, the absorbent gelling material serves the function of gradually supplying water to the iron powder component, wherein the water is released at a controlled rate. Nonlimiting examples of suitable absorbent gelling materials include those absorbent gelling materials that have fluid-absorbing properties and can form hydrogels upon contact with water. One specific example of such an absorbent gelling material is the hydrogel-forming, absorbent gelling material that is based on a polyacid, for example polyacrylic acid. Hydrogel-forming polymeric materials of this type are those which, upon contact with liquids such as water, imbibe such fluids and thereby form the hydrogel. These preferred absorbent gelling materials will generally comprise substantially water-insoluble, slightly cross-linked partially neutralized, hydrogel-forming polymer materials prepared from polymerizable, unsaturated, acid-containing monomers. In such materials, the polymeric component formed from unsaturated, acid-containing monomers may comprise the entire gelling agent or may be grafted onto other types of polymer moieties such as starch or cellulose. Acrylic acid grafted starch materials are of this latter type. Thus, specific suitable absorbent gelling materials include hydrolyzed acrylonitrile grafted starch, acrylic acid grafted starch, polyacrylate, maleic anhydride-based copolymer, and combinations thereof. The polyacrylates and acrylic acid grafted starch materials are preferred. Nonlimiting examples of commercially available polyacrylates include those polyacrylates which are available from Nippon Shokubai located in Chattanooga, Tenn. (U.S.A.).

The absorbent gelling material has a median particle size of from about 300 μm to about 800 preferably from about 400 μm to about 800 more preferably from about 500 μm to about 800 Absorbent gelling materials having a median particle size of 300 μm or greater have been shown to contribute to minimal or no segregation effects.

In addition to the absorbent gelling material, the particulate exothermic compositions of the present invention can optionally comprise other water-holding materials that have capillary function and/or hydrophilic properties. These optional water-holding materials can be included in the particulate exothermic compositions at concentrations ranging from about 0.1% to about 25%, preferably from about 0.5% to about 20%, more preferably from about 1% to about 15%, by weight of the composition. Nonlimiting examples of such optional water-holding materials include vermiculite, porous silicates, wood powder, wood flour, cotton, paper, vegetable matter, carboxymethylcellulose salts, inorganic salts, and mixtures thereof. The absorbent gelling material and optional water-holding materials are further described in U.S. Pat. Nos. 5,918,590 and 5,984,995; which descriptions are incorporated by reference herein.

### Metal Salt

The particulate exothermic composition of the present invention comprises one or more metal salts at concentrations ranging from about 0.5% to about 10%, preferably from about 0.5% to about 7%, more preferably from about 1% to about 5%, by weight of the composition.

The metal salts suitable for use herein include those metal salts that serve as a reaction promoter for activating the surface of the iron powder to ease the oxidation reaction with air and provide electrical conduction to the exothermic composition to sustain the corrosive reaction. In general, several suitable alkali, alkaline earth, and transition metal salts exist which can be used, alone or in combination, to sustain the corrosive reaction of iron.

Nonlimiting examples of suitable metal salts include sulfates, chlorides, carbonate salts, acetate salts, nitrates, nitrites, and mixtures thereof. Specific nonlimiting examples of sulfates include ferric sulfate, potassium sulfate, sodium sulfate, manganese sulfate, magnesium sulfate, and mixtures thereof. Specific nonlimiting examples of chlorides include cupric chloride, potassium chloride, sodium chloride, calcium chloride, manganese chloride, magnesium chloride cuprous chloride, and mixtures thereof. Cupric chloride, sodium chloride, and mixtures thereof are the preferred metal salts. An example of a commercially available sodium chloride includes the sodium chloride available from Morton Salt located in Chicago, Ill. (USA).

### Water

The particulate exothermic compositions of the present invention comprise water at concentrations ranging from about 1% to about 35%, preferably from about 5% to about 33%, by weight of the composition. The water suitable for use herein can be from any appropriate source. For example, tap water, distilled water, or deionized water, or any mixture thereof, is suitable for use herein.

It is known that the thermal performance of heat cells is highly sensitive to moisture level, and a typical heat cell can comprise water concentrations at or above about 27% to sustain the heating temperature of the heat cell. However, the inclusion of high concentrations of water at levels of about 27% or above can result in slower than desired initial heating temperatures. Therefore, the ability to rapidly reach the desired temperature for a therapeutic benefit and the ability to sustain the temperature are critical. This goal can be achieved by incorporating a sufficient weight ratio of water to absorbent gelling material or other water-holding materials such that the particulate exothermic compositions have a high internal water retention and high interstitial particle void volumes. The particulate exothermic compositions of the present invention comprise a weight ratio of water to absorbent gelling material or other water-holding materials of from about 3:1 to about 9:1, preferably from about 4:1 to about 7:1, by weight of the exothermic composition.

### Other Optional Components

The exothermic compositions of the present invention may further comprise one or more other optional components known or otherwise effective for use in exothermic compositions, provided that the optional components are physically and chemically compatible with the compositional components described hereinabove, or do not otherwise unduly impair product stability, aesthetics, or performance. Other optional components suitable for use herein include materials such as agglomeration aids including corn syrup, maltitol syrup, crystallizing sorbitol syrup, and amorphous sorbitol syrup; dry binders including microcrystalline cellulose, microfine cellulose, maltodextrin, sprayed lactose, co-crystallized sucrose and dextrin, modified dextrose, mannitol, pre-gelatinized starch, dicalcium phosphate, and calcium carbonate; oxidation reaction enhancers including elemental chromium, manganese, copper, and compounds comprising said elements; hydrogen gas inhibitors including inorganic and organic alkali compounds, and alkali weak acid salts, specific nonlimiting examples include sodium thiosulfate, sodium sulfite, sodium hydroxide, potassium hydroxide, sodium hydrogen carbonate, sodium carbonate, calcium hydroxide, calcium carbonate, and sodium propionate; fillers such as natural cellulosic fragments including wood dust, cotton linter, and cellulose, synthetic fibers in fragmentary form including polyester fibers, foamed synthetic resins such as foamed polystyrene and polyurethane, inorganic compounds including silica powder, porous silica gel, sodium sulfate, barium sulfate, iron oxides, and alumina; anti-caking agents such as tricalcium phosphate and sodium silicoaluminate; and mixtures thereof. Such components also include thickeners such as cornstarch, potato starch, carboxymethylcellulose, and alpha-starch, and surfactants such as those included within the anionic, cationic, nonionic, zwitterionic, and amphoteric types. Still other optional components may be included within the compositions or articles herein, as appropriate, including extending agents such as metasilicates, zirconium, and ceramics, and mixtures thereof. The other optional components can be included in the particulate exothermic compositions at concentrations ranging from about 0.01% to about 35%, preferably from about 0.1% to about 30%, by weight of the composition.

### Method of Manufacture

The particulate exothermic compositions of the present invention may be prepared by any known or otherwise effective technique suitable for providing an exothermic composition that provides a therapeutic heat benefit. The particulate exothermic compositions of the present invention are preferably prepared using conventional blending techniques. Suitable methods of blending the components of the particulate exothermic compositions of the present invention are more fully described in U.S. Pat. No. 4,649,895 to Yasuki et al., issued Mar. 17, 1987, which descriptions are incorporated by reference herein.

A typical technique of blending the components of the particulate exothermic compositions involve adding carbon to a blender or mixer, followed by adding a small amount of the total water, and then mixing the carbon/water combination. Usually enough water is added to assist in blending while avoiding escalated corrosion. Mixing is stopped and an absorbent gelling material or other water-holding materials is added to the carbon/water combination. Mixing is resumed until all the components are mixed thoroughly, and then iron powder is added and mixed. The composition is then blended until thoroughly mixed to form a particulate pre-mix. Sodium chloride, optionally an hydrogen gas inhibitor such as sodium thiosulfate, and the remaining water are separately mixed to form a brine solution which is then added to the iron powder pre-mix to form a particulate exothermic composition that is used in the construction of a heat pathway of the present invention.

Individual heat pathways can typically be prepared by adding a fixed amount of the particulate pre-mix composition to a pocket in a film layer substrate sheet such as the pocket in a polypropylene nonwoven/LDPE film layer substrate sheet. In this process, water or brine is rapidly dosed on top of the pre-mix composition, and a flat sheet of a polypropylene nonwoven/poly(ethylene-vinyl acetate) film layer substrate is placed over the pathway with the poly(ethylene-vinyl acetate) film side facing the LDPE film side of the preformed pocket containing sheet. The film layers of the two sheets are bonded together using a low heat, forming a unified structure. The resulting heat cell contains the particulate exothermic composition sealed in the pocket between the two film layer substrate sheets.

Alternatively, individual heat cells can be prepared by using vacuum to form a pocket. That is, vacuum is used to draw the film layer substrate surface into a mold as the particulate pre-mix composition is placed on top of the film layer substrate surface directly over the mold. The particulate pre-mix composition drops into the vacuum formed pocket and is held in place by the vacuum exerted upon the particulate pre-mix composition in the bottom of the mold. Next, a brine solution is rapidly dosed on top of the pre-mix composition. A second film layer substrate surface is then placed over the first film layer substrate surface, such that the particulate exothermic composition is between the two surfaces. The particulate exothermic composition is then sealed between sealing layers 30 or elongated elastic layers 345, 346 to form a tube or welded heating area.

As a result of the exothermic material 10 and 301, for example, having a negative (violent) reaction to RF frequency, it is important that during the assembly process, that the exothermic material 10 and 310 does not come in contact with the RF welding tool. On alternative is to now conduct 90 to 99% of the welding prior of the exothermic compound sealing layers 30 or elongated elastic layers 345, 346 prior to the loading of the exothermic material 10 and 310. A small loading port, perhaps ¼″ or so, will remain un-welded. The resulting tube or welded heating area will be injected with exothermic compound through this small port by a feeding tube. The feeding tube can then be removed and the port opening can be welded or heat-sealed closed. Complicating the loading of the preferred tapes 100, 400 or wraps are the “blocking” lines or intermediate movement blocking surfaces, such as the seams or bonds 410 shown in FIG. 12, that we are using in order to impede the movement of the exothermic material within the tapes 100, 400 or wrap. In order to aid with this process, pressurized argon or nitrogen can be introduced into the small port to expand the tube or welded heating area right before the exothermic material is introduce. The argon or nitrogen can be squeezed out after the tube or welded heating area is full.

The resultant heat cells or compartments 414 can be used alone, or as a plurality of heat pathways or heated areas, and the heat cells or compartments 414 can be incorporated into various disposable heating devices such as disposable tapes and body wraps. Typically, the body wraps have a means for retaining the wraps in place around various parts of the body, such as knee, neck, back, etc. and can comprise any number of styles and shapes, wherein the retaining means include a fastening system such as a reclosable two-part hook and loop fastening system.

The resultant tapes or wraps are alternatively packaged in a secondary air-impermeable package to prevent the oxidation reaction from occurring until desired as described in the aforementioned U.S. Pat. No. 4,649,895, incorporated herein by reference. Alternatively, air impermeable removable adhesive strips can be placed over the aeration holes in the tapes and wraps such that, when the strips are removed, air is allowed to enter the heat cell, thus activating the oxidation reaction of the iron powder.

### Possible Particulate Exothermic Compositions

| | Example 1 | Example 2 | Example 3 |
|---|---|---|---|
| Component | (Wt. %) | (Wt. %) | (Wt. %) |
| Iron powder | 60.40-65.49 | 56.75-61.53 | 58.70-63.64 |
| Activated Carbon | 4.05 | 3.81 | 3.94 |
| (Optional Absorbent | 0-5.09 | 0-4.78 | 0-4.94 |
| Gelling Material) | | | |
| Sodium Chloride | 3.02 | 3.47 | 1.38 |
| Sodium Thiosulfate | 0.38 | 0.43 | — |
| Water | 27.06 | 30.76 | 31.04 |

See formulations in U.S. Pat. Nos. 7,878,187; 4,366,804; 4,649,895; 5,046,479 and Re. 32,026 which are hereby incorporated herein by reference

### EXAMPLES

An exothermically heated elastic tape was constructed using a laminate of TPU having disposed there between a layer of exothermic material (⅛th inch- 3/16th inch in thickness). Each layer of TPU (2 layers total) was approximately 0.015 mm in thickness. Typically, kinesiology tape employs acrylic-based or silicone-based adhesive that is sprayed on one or more layers, primarily to adhere the kinesiology tape to the skin. A separate tape/bag construction was manufactured using conventional kinesiology cotton fabric. Both constructions used acrylic-based adhesive on two-sided transfer tape. In the TPU example, the top layer of TPU was micro-perforated mechanically with a perforation roll. Preferably an array is selected from about 20 to about 60 pins/cm2, with, e.g., tapered points and diameters of from about 0.2 mm to about 2 mm, preferably from about 0.4 mm to about 0.9 mm. The bottom TPU layer of the TPU embodiment was not perforated. The cotton embodiment included porosity in the cotton weave, but no hole punching.

### Results

The cotton bag, when exposed to air, generated heat for a shorter duration (than the TPU assembled bag) and the top cotton layer became wet. The action of the exothermic material ended prematurely. On the other hand, the TPU-layered bag construction maintained its heat for a longer period of time at relatively higher temperatures.

Various thickness layers of TPU were employed in multiple combinations of bag constructions which showed that the range of about 0.01-0.25 mm, more preferably about 0.015 mm of TPU provided an oxygen/air and water vapor breathable substrate, even when perforated, but was generally waterproof, or resulted in a barrier which prevented water in the exothermic material from leaking, and prevented water from outside of the tape to penetrate to the skin of the wearer. The preferred TPU tape-based construction should have a stretch of at least about 10%-90%, preferably about 65%, and a recovery of 10-99%, preferably about 90% or better.

Further, it was observed that when silicone or acrylic-based adhesives were adhered to the TPU layer directly, as a bottom layer, there was no residue on the skin of the wearer when the tape was removed, and there was greater adherence of the skin to the TPU bottom layer. With the acrylic-based adhesive systems being believed to be more adherent to the bottom TPU layer than to the average human skin, the tape could remain in place during strenuous athletic performances, such as triathlons, or marathons, where the wearer might expose the tape to excessive movement, aqueous water, and plenty of perspiration. In addition, it was further noticed that the use of a 50% polyester/50% nylon top layer provided an ideal complement above the perforated TPU layer, and held the heat of the exothermic reaction for a longer period of time, while also preventing wicking of liquid water from the exothermic composition. This is due to the non-wicking nature of the polyester combined with the heat insulating properties of the nylon in the fabric.

Experiments in the number and size of the perforations in the top TPU layer were also conducted. Bigger holes and/or greater densities resulted in higher temperatures from a given quantity of exothermic material for a shorter period of time, while smaller holes and smaller densities of holes resulted in less heat from the exothermic material, for longer durations.

It is further expected that adhesive layers for bonding components to one another can be replaced with heat seals, or welded seals such as RF welding or microwave solutions deposited on the layers, which are deposited on those surfaces before contact with another layer and then microwaving the composite. It was also observed that the 50% polyester/50% nylon top layers provided excellent surfaces for screen printing, 3D printing, e.g., for urethane or silicone patterns.

1. A tape or wrap comprising:
first and second elongated elastic layers sized to conform to the shape of a portion of the external skin of the body of a wearer; and
a heated area comprising an exothermic material sandwiched between said first and second elastic layers, wherein said exothermic material is activated by exposing said exothermic material to oxygen;
wherein said tape or wrap has a elasticity of at least about 10-90% and said heated area is capable of substantially expanding and contracting with the tape or wrap.

2. The tape or wrap of claim 1, further comprising a plurality of intermediate movement blocking surfaces disposed in a space formed between said first and second elongated elastic layers, and disposed to resist said exothermic material from accumulating or clumping at one or both ends of said tape or wrap during use.

3. The tape or wrap of claim 2, wherein said intermediate movement blocking surfaces comprise a bond formed between the first and second elongated elastic layers or a plurality of raised or formed surfaces on the first elongated elastic layer, second elongated elastic layer, or both, or another layer, such as a grid, mesh, netting, or maze-like surface, disposed between the elongated elastic layers; which intermediate movement blocking surfaces help to impede the movement of said exothermic material during use, but which allow some movement of said exothermic material around at least one of said intermediate movement blocking surfaces during use.

4. The tape or wrap of claim 1, wherein said exothermic material contained within said heated area has a first thickness when said tape or warp is unstretched and a second thickness when said tape or wrap is stretched, said second thickness being less than said first thickness, whereas said exothermic material contained within said heated area having the second thickness is capable of generating a surface temperature of about 32° C. to about 70° C. (89.6-158° F.).

5. The tape or wrap of claim 1, wherein exothermic material comprises: iron powder, water, and a carbon-containing material in loose particulate form.

6. The tape or wrap of claim 1, wherein a second of said elongated elastic layers is permeable to gaseous oxygen and resistant to liquid water.

7. The tape or wrap of claim 1, wherein a first of said elongated elastic layers is perforated to provide enough oxygen in an ambient environment to permit said exothermic compound to exothermically react to generate a temperature of at least about 100 F (37.8 C) for at least about 30 minutes.

8. The tape or wrap of claim 1, wherein said first and second elongated elastic layers comprise a non-woven polymeric film having a Tensile Strength of about 2000-10000 psi, a Stress at 100% elongation of about 200-3000 psi, a Tear Strength of about 100-1000 lbf/in; and a Glass Transition Temperature of about −100 F-+10 F.

9. A tape or wrap sized to conform to a portion of the external skin of the body of a wearer, said tape or wrap having a longitudinal length, a width and at least two transverse ends, comprising:
first and second elongated elastic layers, each of said elastic layers comprising a thermoplastic polyurethane (“TPU”) layer having a thicknesses of no greater than about 0.001-1.5 mm; said first and second elastic layers comprising at least one peripheral bond and a plurality of intermediate movement blocking surfaces disposed in a space formed between said first and second elongated elastic layers;
an exothermic material sandwiched between said first and second elastic layers and also at least between a first pair of said plurality of intermediate movement blocking surfaces, said first pair of said plurality of intermediate movement blocking surfaces at least partially impeding the movement of exothermic material when worn by a user; and wherein said exothermic material is activated by exposing said exothermic material to oxygen.

10. The tape or wrap of claim 9, wherein said tape or wrap can stretch at least about 10-90% of its original length which provides for sufficient skin contact in order to optimize heat transfer.

11. The tape or wrap of claim 9, wherein when said tape or wrap is stretched greater than its normal length, and then adhesively applied to the skin of a wearer, so it will recoil and create a pulling force on the skin.

12. The tape or wrap of claim 9, wherein said tape or wrap can stretch up to 20-70% of its original length in the warp direction.

13. The tape or wrap of claim 9, wherein said tape or wrap can stretch up to about 10%-90% of an original dimension of said tape or wrap in any direction.

14. The tape or wrap of claim 9, wherein said heated area is capable of substantially expanding and contracting with the tape or wrap.

15. The tape or wrap of claim 9, wherein at least a first one of said first and second elongated elastic layers is micro-perforated for controlling a heating temperature and a duration of an exothermic reaction of said exothermic material.

16. A tape or wrap, which is sized to conform to a portion of the external skin of the body of a wearer when worn, comprising:
an exothermic compound layer disposed between a first and second exothermic compound sealing layer to form a heated area substantially along the length of said tape or wrap; and
said first exothermic compound sealing layer being bonded to a top elastic fabric layer, and second exothermic compound sealing layer being bonded to a second elastic fabric layer, whereby the exothermic compound layer disposed between said first and second exothermic compound sealing layers expand substantially proportionately in length to the tape or wrap as the tape or wrap is stretched.

17. A tape or wrap which is sized to conform to a portion of the external skin of the body of a wearer, comprising: an exothermic compound layer disposed between a pair of elastic fabric layers forming a heated area having an elasticity of about 10%-90% in any or all directions.

18. The tape or wrap of claim 17, wherein each of said pair of elastic fabric layers comprises a TPU film or coating having a thickness of less than about 0.02 mm.

19. The tape or wrap of claim 18, wherein said exothermic compound layer disposed between a pair of elastic fabric layers is then disposed between a pair of breathable insulating fabric layers also having an elasticity of about 10%-90% at least in the warp direction, whereby said exothermic compound layer disposed between a pair of elastic fabric layers is capable of expanding and contracting with the elasticity of the tape or warp without substantially impeding same.

20. The tape or wrap of claim 19, wherein said each of said pair of elastic fabric layers comprises a TPU layer and said breathable insulating fabric layers are selected from: knitted or woven fabrics.

21. The tape or wrap of claim 20, wherein said knitted or woven fabrics comprise fibers selected from the group consisting essentially of: cotton, spandex, rayon, nylon, polyester, or a combination thereof.

22. The tape or wrap of claim 20, wherein the primary means for fastening the tape or wrap to a wearer comprises: adhesive pads which can be applied to the underside of the wrap and then adhered to the skin of a wearer, an adhesive tape, elastic or mechanical straps, hook and loop materials, or compression wear.

23. A method of treating a wearer for muscle pain or injury, comprising:
(a.) providing an elastic tape sized to conform to a portion of the external skin of the body of a wearer, comprising: an exothermic compound layer disposed between a pair of elastic fabric layers having an elasticity of about 10%-90% in any or all directions, and an adhesive backing layer;
(b.) stretching said elastic tape about 10%-90% of its original length;
(c.) adhering said adhesive backing layer of said adhesive tape to the skin of a wearer proximate to said muscle pain or injury while said elastic tape if stretched; and
(d.) simultaneously providing heat therapy and a kinesiologic effect on said skin of said wearer proximate to said muscle pain or injury.
